(19) United States
(12) Patent Application Publication
(10) Pub. No.: US 2026/0130952 A1
(43) Pub. Date: May 14, 2026

(54) ENGINEERED BACTERIA AND METHODS OF USE IN TUMOR REMODELING

(71) Applicant: University of Cincinnati, Cincinnati, OH (US)

(72) Inventors: Nalinikanth Kotagiri, West Chester, OH (US); Shindu C. Thomas, Cincinnati, OH (US)

(73) Assignee: University of Cincinnati, Cincinnati, OH (US)

(21) Appl. No.: 18/704,590

(22) PCT Filed: Oct. 27, 2022
(86) PCT No.: PCT/US2022/078765

§ 371 Date: Apr. 25, 2024

Related U.S. Application Data
(60) Provisional application No. 63/272,430, filed on Oct. 27, 2021.

(51) Int. Cl. A61K 35/74; A61K 40/11; A61K 40/15; A61K 40/31; A61K 45/06; A61P 35/00; C12N 1/20; C12N 9/26

(52) U.S. Cl. CPC A61K 35/74; A61K 40/11; A61K 40/15; A61K 40/31; A61K 45/06; A61P 35/00; C12N 1/20; C12N 9/2474; C12Y 302/01035; C07K 2319/55

(57) ABSTRACT

Provided herein is a hypervesiculating Escherichia coli Nissle (ΔECHy) bacterium engineered to produce outer membrane vesicles (OMVs), said OMVs packaging a fusion peptide including cytolysin A (ClyA) and hyaluronidase (Hy). Also provided are methods of remodeling a tumor, methods of treating cancer, and pharmaceutical compositions including the engineered ΔECHy bacterium.

### CROSS-REFERENCE TO RELATED APPLICATION

This application claims priority to U.S. Provisional Application Ser. No. 63/272,430, filed Oct. 27, 2021, the entire contents of which are incorporated herein by reference.

### TECHNICAL FIELD

The present disclosure relates to the fields of microbiology and cancer therapy. Specifically, the present disclosure relates to engineered hypervesiculating Escherichia coli expressing confined cytolysin A-hyaluronidase (CHy) and its use in tumor remodeling.

### BACKGROUND

Desmoplastic solid tumors are characterized with the rapid build-up of extracellular matrix macromolecules such as hyaluronic acid (HA). The resulting physiological barrier prevents the infiltration of immune cells and impedes the delivery of anticancer agents.

Solid tumors display a vast heterogeneous milieu that consists of epithelial cells, mutated highly proliferative epithelial cells, abnormal blood vessels, fibroblasts, cancer associated fibroblast (CAFs), proteins, proteoglycans and glycosaminoglycans, all densely swamped in a sea of interstitial fluid. Another significant hallmark of solid tumors is the development of an unorganized network of compressed and highly permeable blood vessels exhibiting abnormal morphology, ultimately resulting in heterogeneous perfusion and deficient drainage. The overgrowth of fibrous connective tissue in a rapidly proliferating tumor matrix works in tandem with poor blood perfusion further impeding the influx and distribution of nutrient supply thereby promoting hypoxic conditions in the tumor microenvironment (TME). The resulting hypoxia has unfavorable therapeutic implications aiding tumor progression by modulating the apoptotic pathways and mitochondrial activity, inducing autophagy and inactivating the tumor suppressive p53 pathway. Abundantly dispersed within the tumor extracellular matrix (ECM) are CAFs that secrete fibrotic macromolecules that result in a highly dense and difficult to navigate extracellular space. This severe desmoplastic reaction in combination with poor perfusion forms a physical barrier hindering tumor access, which is a major cancer cell resistance mechanism inhibiting the infiltration of cancer-cell eliminating immune cells and therapeutic agents, particularly large macromolecules, such as therapeutic antibodies and nanoparticles. Adhesion of cancer cells to ECM elements is essential for their survival and ECM macromolecules have been demonstrated to promote drug resistance via multiple biological mechanisms. Hyaluronic acid (HA), which is a glycosaminoglycan secreted by stromal CAFs, has been implicated in mediating resistance to tyrosine kinase inhibitors (TKIs), such as lapatinib, by reducing its accumulation and improving the apoptotic threshold of cancer cells. Modulating and engineering the tumor stroma to penetrate this complex desmoplastic barrier is critical for efficient drug delivery and successful immuno- and chemotherapies.

Targeting ECM macromolecules via CAF inhibition or enhancing their degradation is one way to improve the penetration of immuno- and chemotherapeutic agents. Presently, there are no approved therapies in the U.S. for specifically targeting the desmoplastic reaction. Clinical trials with small molecules such as calcipotriol, nintedanib and metformin that target CAFs are ongoing. These molecules act on the CAFs and inhibit the synthesis of extracellular macromolecules. An alternative approach is to enhance the degradation of these macromolecules using biologies such as the oncolytic adenovirus VCN-01 carrying the hyaluronidase (Hy) gene. Live biotherapeutics in the form of programmable bacteria offer unique advantages over other nanoparticulate dosage forms that can be leveraged for delivering recombinant proteins, thereby modulating molecular mechanisms in vivo and altering cancer cell proliferation and disease progression.

A highly significant amount of intravenously administrated conventional nanoparticulate carriers have been shown to be rapidly eliminated by the hepatobiliary and renal route of systemic elimination. Anaerobic bacteria have been found to localize and multiply in the TME by taking advantage of the leaky tumor vasculature, immunodeficient TME, and the hypoxia associated tumor-tropism. Even a minute fraction of bacterial dose reaching the tumor is sufficient to repopulate and allow therapeutic concentrations of peptide drug to be maintained for a longer period of time. Moreover, selecting a commensal or a probiotic bacterial strain will help in ensuring biocompatibility. Escherichia coli Nissle (EcN) has been historically used as an oral probiotic and has also entered clinical trials for delivering recombinant proteins in vivo. The facultative anaerobic nature and amenability for genetic modification allows EcN to be used as a carrier for targeting hypoxic tumors and delivering peptide based drugs in vivo.

A need exists for new compositions that traverse the physiological barrier of solid tumors and permit delivery of anticancer agents.

### SUMMARY

The present disclosure pertains to an Escherichia coli Nissle bacterium (EcN) engineered to generate outer membrane vesicles (OMVs), wherein the OMVs package a fusion peptide comprising cytolysin A and hyaluronidase (ΔECHy). The engineered ΔECHy bacterium is hypervesiculating and produces OMVs that degrade extracellular tumor barriers and permit infiltration of anticancer agents, thereby promoting tumor remodeling and tumor cancer cell death.

Accordingly, in one embodiment, a hypervesiculating Escherichia coli Nissle (ΔECHy) bacterium engineered to produce outer membrane vesicles (OMVs) is provided herein, said OMVs packaging a fusion peptide comprising cytolysin A (ClyA) and hyaluronidase (Hy).

In another embodiment, a method of remodeling a tumor is provided, the method comprising contacting the tumor with a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).

In another embodiment, a method of treating a solid tumor in a subject in need thereof is provided, the method comprising administering to the subject an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).

In another embodiment, a pharmaceutical composition is provided, the composition comprising: an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy); and at least one pharmaceutically-acceptable carrier.

These and other objects, features, embodiments, and advantages will become apparent to those of ordinary skill in the art from a reading of the following detailed description and the appended claims.

### BRIEF DESCRIPTION OF THE DRAWINGS

FIG. 1 is an illustration depicting an untreated tumor having an extracellular matrix (ECM) that inhibits penetration of infiltrating immune cells into the tumor microenvironment (left panel); and a tumor treated with ΔECHy bacteria, which produce outer membrane vesicles (OMVs) confined CHy that decreases tumor tissue HA and improves penetrability of anticancer agents.

FIG. 2A is a schematic showing deletion of nlpI gene using homologous recombination.

FIG. 2B is a TEM image of ΔE and isolated ΔE OMVs showing nanosized vesicles (black bar=1 μm).

FIG. 2C is a TEM images of ΔE and isolated ΔE OMVs showing nanosized vesicles (black bar=200 nm).

FIG. 2D is a plot depicting the size analysis of ΔE derived OMVs via DLS. A nanoparticle population with moderate polydispersity was obtained.

FIG. 3A is a schematic showing translocation of CHy fusion protein to the bacterial outer membrane and vesiculation to form OMVs.

FIG. 3B and FIG. 3C are images of immunoblots for detecting the presence of CHy protein (56 kDa). ΔE lysate was used as the negative control and ΔECHy lysate and ΔECHy OMVs were analyzed for the presence of CHy.

FIG. 3D is an image showing qualitative analysis for Hy activity using the HA agarose plate assay. Zones of degradation were observed around the ΔECHy bacteria while ΔE showed no activity.

FIG. 3E and FIG. 3F are graphs showing quantitative estimation of Hy activity using the HA degradation spectrophotometric assay for three different concentration of HA (0.1, 0.2, 0.4 μg/ml). Significant reduction in HA optical density was observed for the ΔECHy group in comparison to ΔE after 6 hours (p<0.0001) and 24 hours (p<0.0002 for 0.1 μg/ml and p<0.0001 for 0.2 and 0.4 μg/ml).

FIG. 4A depicts bioluminescence images showing (n=5) EcN-luxCDABE accumulation in 4T1 tumors in BALB/cJ mice after intravenous injection at the end of 24 hours (left image); ΔElux accumulation in 4T1 tumors in BALB/cJ mice after intravenous injection at the end of 24 hours (center image); and ΔElux accumulation in MC38 tumors in C57BL/6 mice after intravenous injection at the end of 24 hours (quantified intensity units in radiance) (right image.

FIG. 4B depicts PCR analysis of tissue samples for detecting pecol19A-ClyA-Hy plasmid fragments in 4T1 tumors (731 bp) (n=4).

FIG. 4C depict PCR analysis of tissue samples for detecting pecol19A-ClyA-Hy plasmid fragments in MC38 tumors (731 bp) (n=4).

FIG. 4D is a schematic depicting the labelling of OMVs with the 64Cu-YbT complex.

FIG. 4E depicts PET/CT images along the longitudinal (left) and transverse (right) axis showing in vivo biodistribution of 64Cu-YbT labelled OMVs and preferential uptake in tumor. Arrow points towards the tumor location (n=3).

FIG. 5A depicts a treatment protocol for the in vivo analysis of HA degradation in 4T1 syngeneic xenografts in BALB/cJ mice.

FIG. 5B is a line plot for different groups showing mean tumor volume over the course of study (n=5). The combination of ΔECHy with lapatinib showed the slowest rate of tumor growth (ΔECHy+lapatinib vs lapatinib comparison using paired t-test, p=0.0078, p-value summary=**).

FIG. 5C depicts Kaplan Meier survival analysis for different groups tested. The combination of ΔECHy with lapatinib (5 mg/Kg) showed significant improvements in survival when compared to the other groups tested. The four groups were compared, and the median survival time was calculated using the log-rank (Mantel-Cox) test, which is indicated for each group (corresponding color-coded numerals) (*p=0.0421).

FIG. 5D provides representative images for IHC analysis of tumor tissues from different treatment groups: saline control, ΔECHy, lapatinib and ΔECHy+lapatinib (inset ×100 and outset ×400). HA synthesis was found to be qualitatively reduced in the ΔECHy treated groups. The combination of ΔECHy+lapatinib showed the most reduction in cellular proliferation and markedly increased cellular apoptosis. Smooth muscle actin, a marker for CAFs was also found to be reduced considerably in this group thereby indicating stromal reengineering which is a favorable response towards cancer therapy. Quantification of markers (arbitrary units) is indicated in yellow digits for each image. Black bar for scale corresponds to 50 μm.

FIG. 6A-FIG. 6D depict line plots and Kaplan Meier survival analysis for different groups combining ΔECHy with PDL1 antibody showing mean tumor volume over the course of study in 4T1 tumors (FIG. 6A-6B) and MC38 tumors (FIG. 6C-6D) (n=5). The combination of ΔECHy with anti-PDL1 in comparison to anti-PDl1 antibody brought about significant regression in tumor growth rate in both the groups. (**p=0.0078 (Wilcoxon matched-pairs signed rank t-test)). However, the combination therapy was only able to significantly improve the median survival in the animals with MC38 tumors. The four groups were compared, and the median survival time was calculated using the Log-rank (Mantel-Cox) test, which is indicated for each group (corresponding color-coded numerals) (**p=0.080).

FIG. 6E provides IHC analysis of tumor tissues from different treatment groups in MC38 tumor models: saline control, ΔECHy, anti-PDL1 and ΔECHy+anti-PDL1 (inset ×100 and outset ×400). Analogous to the results in the 4T1-lapatinib study, HA synthesis was again found to be qualitatively reduced in the ΔECHy treated groups. The combination of ΔECHy with the anti-PDL1 antibody showed a reduction in cellular proliferation and markedly increased cellular apoptosis. CAFs were also found to be reduced in this group thereby indicating stromal reengineering. Quantification of markers (arbitrary units) is indicated in digits for each image. Black bar for scale corresponds to 50 μm.

FIG. 7A is an image showing mice distribution study for ΔElux showing no detectable signals in-vivo (n=4).

FIGS. 7B-7D are images showing PCR amplification for detecting the presence of ΔElux containing pakfplux1 plasmid from the microbiome genetic material isolated from each organ (121 bp) at 4 hours (FIG. 7A), 24 hours (FIG. 7C), and 49 hours (FIG. 7D).

FIG. 7E is a graphical plot of cytokine profiling results from the biocompatibility study in mice. ΔECHy was compared with saline and only two cytokines out of the 40 tested were observed to be significantly regulated, complement component 5a (C5a, p=0.016) was downregulated and the tissue inhibitor of metalloproteinases (TIMP-1, p<0.0001) was upregulated. No other inflammatory markers were detected (n=3).

FIG. 8 is an image of colony PCR results showing the presence of the amplified chloramphenicol from the ΔE colonies (500 bp).

FIG. 9 is a schematic of the expression plasmid pecol19A-ClyA-Hy.

FIG. 10 is a plot showing ΔECHy and EcN growth curves (Optical Density at 600 nm vs time (hours)).

FIG. 11 provides IHC analysis of breast and pancreatic tumor tissues showing abundant extracellular macromolecules: HA, Fibronectin, Periostin and Collagen IV (×400 magnification).

FIG. 12 depicts line graphs showing tumor progression in mice with 4T1 tumors for each treatment group tested.

FIG. 13A is a graph showing 4T1-IHC marker quantification for treatment groups HA and SMA. (**** for p<0.0001, ***for p<0.001, ** for p<0.01).

FIG. 13B is a graph showing 4T1-IHC marker quantification for treatment groups CC3 and Ki67. (**** for p<0.0001, ***for p<0.001, ** for p<0.01).

FIG. 14 depicts line graphs showing tumor progression in mice with MC38 tumors for each treatment group tested.

FIG. 15A is a graph showing MC38-IHC marker quantification for treatment groups HA and SMA. (**** for p<0.0001, ***for p<0.001, ** for p<0.01).

FIG. 15B is a graph showing MC38-IHC marker quantification for treatment groups CC3, Ki67, and CD8. (**** for p<0.0001, ***for p<0.001, ** for p<0.01).

FIG. 16A is a diagram mapping the location of different spots representing cytokine specific antibodies in a proteome profiling study of the regulation of cytokines and chemokines in mice (plasma).

FIG. 16B provides images of nitrocellulose membrane for each group tested (C1, C2, C3, ΔECHy1, ΔECHy2, ΔECHy3).

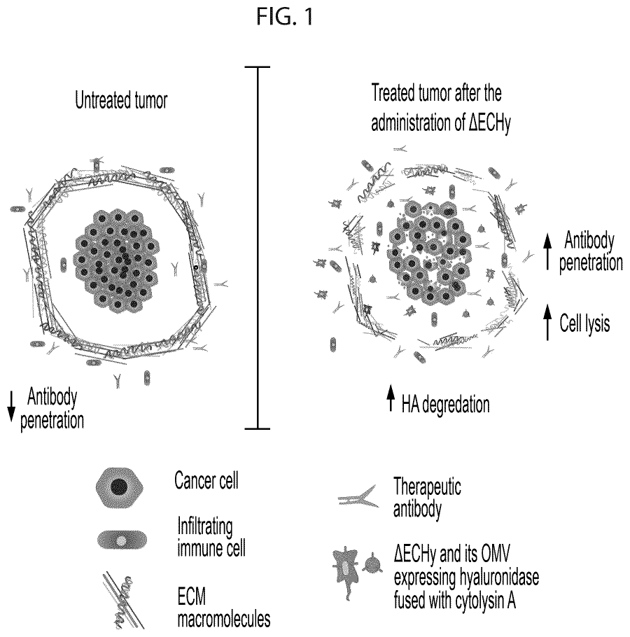

### DETAILED DESCRIPTION

The following description of particular embodiment(s) is merely exemplary in nature and is in no way intended to limit the scope of the invention, its application, or uses, which may vary. The invention is described with relation to the non-limiting definitions and terminology included herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of components, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.10% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all terms (including technical and scientific terms) used herein have the same meaning as commonly understood by one of ordinary skill in the art to which this disclosure belongs. It will be further understood that terms such as those defined in commonly used dictionaries should be interpreted as having a meaning that is consistent with their meaning in the context of the relevant art and the present disclosure, and will not be interpreted in an idealized or overly formal sense unless expressly so defined herein.

The term “subject,” as used herein, refers to any mammalian subject, including mice, rats, rabbits, pigs, monkeys, humans, and the like. In a specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is cancer.

The present disclosure is related to an engineered hypervesiculating ΔECHy strain capable of overproduction of nanoscale OMVs in situ after localizing within tumors. The ΔECHy bacteria are programmed to express Hy enzyme for degrading HA and ClyA for cytolytic activity. The ΔECHy bacteria were phenotyped to produce large quantities of OMVs with functional Hy enzyme. Designing a fusion protein with ClyA serves a dual purpose of anchoring Hy and also promoting cancer cell death. The strain was combined with tyrosine kinase inhibitors and immune checkpoint antibodies to show that ΔECHy can remodel the tumor stroma and induce cancer cell killing that ultimately results in the improvement immunotherapy outcomes and enhancing the activity of tyrosine kinase inhibitors. The biocompatibility of ΔECHy was investigated in vivo by examining the cytokine and chemokine profile to show that the bacteria elicit no inflammatory or immune responses and represents a novel biotherapeutic for in vivo cancer treatment.

### Hypervesiculating ΔECHy Bacteria

In one embodiment, provided is a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).

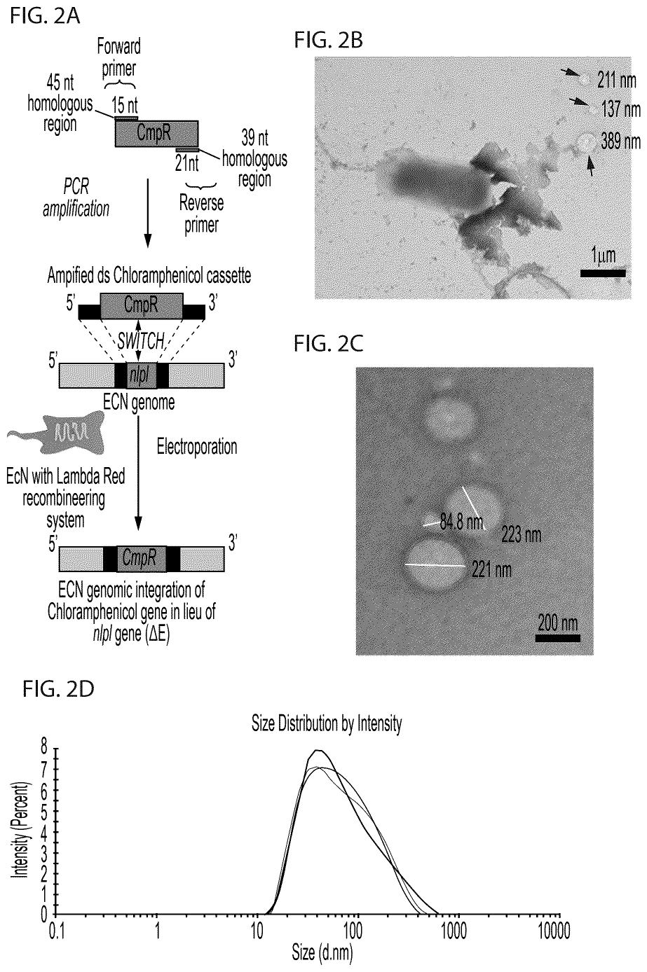

In embodiments, hypervesiculating E. coli strains produce higher OMV yields compared with non-hypervesiculating strands. In embodiments, a hypervesiculating E. coli Nissle (ΔE) strain is developed by deleting the nlpI gene and replacing it with a chloramphenicol cassette. For example, and as demonstrated in FIG. 2A, E. coli Nissle expressing the k-red recombination system proteins, gamma, exo and beta, can be electroporated with a double stranded DNA encoding the chloramphenicol resistance cassette gene with overhangs homologous to the gene sequences upstream and downstream of the nlpI gene. The k-red recombineering system excises the nlpI gene from the genomic DNA and replaces it with the electroporated dsDNA containing the chloramphenicol cassette.

Confirmation of integration of the chloramphenicol cassette can be performed by any known method, including but not limited to polymerase chain reaction, Sanger sequencing, growth on antibiotic resistant plates, and the like.

In embodiments, hypervesiculating E. coli strains can be modified to produce peptides for therapeutic uses. In embodiments, hyaluronidase or enzymatically active fragments thereof, is the peptide for therapeutic use. Hyaluronidase is a protein that has been employed for a variety of therapeutic indications. However, for complete catalytic activity, the hyaluronidase requires post-translational modifications. Without the modifications, the enzyme performs suboptimally, particularly when cloned in bacteria. Accordingly, the present investigators sought to find an alternate form of hyaluronidase of bacterial origin that did not require post-translational modifications.

In embodiments, the hyaluronidase or enzymatically active fragment thereof has a molecular weight of less than 25 kDa. In embodiments, a 217 amino acid sequence of hyaluronidase from Streptomyces koganeiensis (UniProtKB #A0A0U2E2J7) is cloned by the hypervesiculating E. coli. In embodiments, the 217 amino acid sequence of hyaluronidase from Streptomyces koganeiensis is cloned by ΔE.

In embodiments, the hypervesiculating E. coli strains can be modified to produce fusion proteins for therapeutic uses. In embodiments, the fusion proteins comprise hyaluronidase or enzymatically active fragments thereof and another protein or fragment thereof to aid in translocating the enzyme to the bacterial outer membrane for subsequent packaging into OMVs. In embodiments, the other protein or fragment thereof does not cause functional loss to the hyaluronidase. In embodiments, the fusion proteins comprise hyaluronidase or enzymatically active fragments thereof and cytolysin A (ClyA). In embodiments, the hyaluronidase is fused at the C-terminal of ClyA.

In embodiments, ClyA is 34 kDa transmembrane pore-forming hemolytic protein (UniProtKB #P77335) and is cytolytic towards mammalian cells and macrophages. In embodiments, ClyA retains its pore forming cytolytic activity even after the addition of peptide fragments on either ends.

In embodiments, the inclusion of ClyA serves multiple purposes. ClyA serves as an anchoring protein for hyaluronidase. Additionally, ClyA can exert cytotoxic effects against cancer cells. In embodiments, a fusion protein for bacterial membrane localization includes a CHy sequence encoding ClyA on the 5′end and Hy on the 3′ end with a TEV cleavage site in the middle having glycine (G₄) spacers/linkers on either side. In some embodiments, the fusion protein comprises a protein sequence having at least 75%, at least 80%, at least 85%, at least 90%, at least 95%, at least 98%, or at least 99% sequence identity to SEQ ID NO: 17.

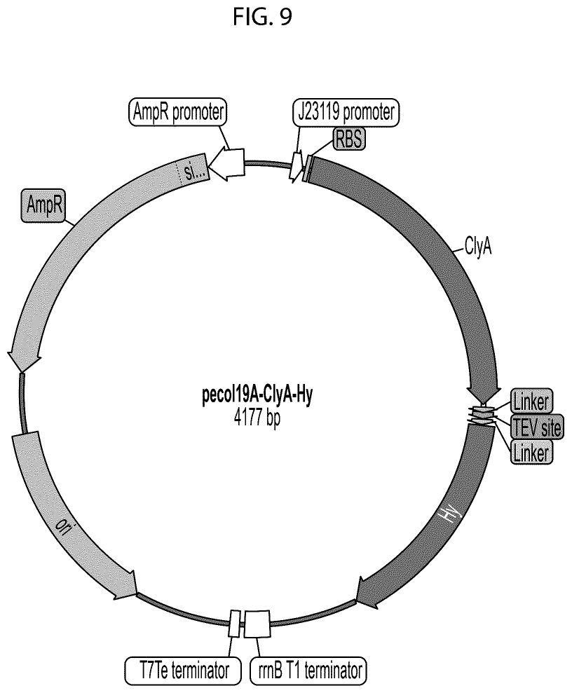

In embodiments, the fusion protein is incorporated into a plasmid. In embodiments, E. coli Nissle codon-optimized gene blocks were obtained for the fusion protein and incorporated into a high copy number plasmid to obtain pecol19A-ClyA-Hy as shown in FIG. 9. In embodiments, the plasmid includes a constitutive promoter to drive production of the fusion protein in the absence of an inducer. In embodiments, the constitutive promoter is selected to have negligible effects on bacterial hosts cellular capacity and growth rate. In embodiments, the constitutive promoter is selected from the group consisting of J23119, J23100, J23108, J23105, J23114, and J23113. In embodiments, the constitutive promoter is J23119.

In embodiments, the assembled plasmid is subcloned into the hypervesiculating E. coli strain. In embodiments, the assembled plasmid is subcloned into ΔE, thereby generating ΔECHy. In embodiments, the OMVs are isolated from ΔECHy, cultured in vitro. The isolated OMVs can be used as an alternative to the ΔECHy bacterium in any embodiments provided herein.

### Methods of Remodeling Tumors and Treating Cancer

In another embodiment, a method of remodeling a tumor is provided, the method comprising contacting the tumor with a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).

In embodiments, the tumor is evaluated for extracellular matrix (ECM) protein composition. Illustrative, non-limiting examples of ECM proteins include HA, periostin, fibronectin and collagen. In embodiments, the tumor is analyzed for fibrotic markers using immunohistochemistry. In embodiments, the tumor is selected from breast, prostate, colon, and pancreatic tumors. In embodiments, the method targets fibrotic and ECM components for stromal reengineering, thereby improving perfusion dynamics and drug delivery kinetics in the tumor.

In another embodiment, a method of treating a cancer in a subject in need thereof is provided, the method comprising administering to the subject an effective amount of ΔECHy bacteria according to any of the embodiments disclosed herein. An “effective amount” is defined herein in relation to the treatment of cancers as an amount that will decrease, reduce, inhibit, or otherwise abrogate the growth of a cancer cell or tumor. In some embodiments, the ΔECHy bacteria can be delivered regionally to a particular affected region or regions of the subject's body. In some embodiments, wherein such treatment is considered more suitable, the ΔECHy bacteria can be administered systemically. For example, the ΔECHy bacteria can be administered orally or parenterally. In a specific embodiment, the ΔECHy bacteria is delivered intravenously.

In embodiments, the method further comprises administering to the subject an effective amount of a second therapeutic agent. In embodiments, the second therapeutic agent is an agent typically administered to treat the symptoms of cancer. In embodiments, the second anticancer agent is selected from the group consisting of tyrosine kinase inhibitors, HER2 inhibitors, EGFR inhibitors, multi-kinase inhibitors, chemotherapy drugs, PARP inhibitors, cancer growth blockers, anti-angiogenics, immune checkpoint antibodies, other monoclonal antibodies, cell based therapies such as CAR-T, NK-T, stem cells, and oncolytic viruses, and combinations thereof. In specific embodiments, suitable tyrosine kinase inhibitors include, but are not limited to lapatinib, gefitinib, erlotinib, pelitinib, CP-654577, CP-724714, canertinib (CI 1033), HKI-272, PKI-166, AEE788, BMS-599626, HKI-357, BIBW 2992, ARRY-380, ARRY-334543, CUDC-101, JNJ-26483327, and JNJ-26483327; and combinations thereof. In specific embodiments, the tyrosine kinase inhibitor is lapatinib.

In embodiments, the second therapeutic agent is a monoclonal antibody. In specific embodiments, the second therapeutic agent is atezolizumab, rituximab, trastuzumab, pertuzumab, trastuzumab emtansine, avelumab, durvalumab, nivolumab, pembrolizumab, cemiplimab, dostarlimab, vopratelimab, spartalizumab, camrelizumab, sintilimab, toripalimab, INCMGA00012, AMP-224, AMP-514, KN035, cosibelimab, AUNP12, CA-170, BMS-986189, combinations thereof, and the like.

In embodiments, the second therapeutic agent is delivered at a dose lower than a therapeutically effective does when administered alone, wherein the ΔECHy bacteria enhances the anti-cancer effect of the second therapeutic agent. The skilled artisan will appreciate that the dosing schedules and amounts set forth herein are exemplary and may be varied by the attending physician in accordance with the age and physical condition of the subject to be treated, the severity of the disease, the duration of the treatment, the nature of concurrent therapy, the particular combination of therapeutic agents being employed, the particular pharmaceutically-acceptable excipients utilized, and like factors within the knowledge and expertise of the attending physician.

In embodiments, the ΔECHy bacterium according to the present disclosure and the second therapeutic agent are co-administered. “Co-administered,” as used herein, refers to administration of the ΔECHy bacterium and the second therapeutic agent such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the ΔECHy bacterium and the second therapeutic agent may be administered concurrently or sequentially.

### Pharmaceutical Compositions

In another embodiment, a pharmaceutical composition is provided, comprising an effective amount of ΔECHy bacteria; and at least one pharmaceutically acceptable carrier.

In embodiments, the ΔECHy are cultured in vitro and the OMVs are isolated and purified using any suitable method. In embodiments, a pharmaceutical composition is provided comprising the isolated OMVs comprising the fusion peptide and a pharmaceutically acceptable carrier.

The pharmaceutically acceptable carrier, or excipient, must be “acceptable” in the sense of being compatible with the other ingredients of the pharmaceutical composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for oral, parenteral, or intratumoral administration, although other specific means of parenteral administration are also viable (such as, for example, intravenous, infusion, intra-arterial, or subcutaneous administration). The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given pharmaceutical composition.

For parenteral administration, suitable compositions include aqueous and non-aqueous sterile suspensions for intramuscular and/or intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, the pharmaceutical composition may be formulated for injection. In other embodiments, the pharmaceutical composition may be formulated for infusion. In a specific embodiment, the pharmaceutical composition is formulated for intratumoral injection, for example, to a malignant tumor.

The term “effective amount,” as used herein, refers to the amount of a composition that is sufficient to achieve a desired biological effect. Generally, the dosage needed to provide an effective amount of the composition will vary depending upon such factors as the subject's age, condition, sex, and other variables which can be adjusted by one of ordinary skill in the art. The compositions of the present disclosure can be administered by either single or multiple dosages of an effective amount. In a specific embodiment, the effective amount is an amount sufficient to degrade the extracellular matrix of a tumor, a tumor barrier, or a tumor microenvironment and permit penetration of anticancer agents. In another specific embodiment, the effective amount is an amount sufficient to promote tumor cell death.

### EXAMPLES

The following examples are given to illustrate various features of the present disclosure and are not intended to be limiting.

### Example 1. Materials and Methods

### Chemicals, Antibodies and Kits

All chemical and reagents used were either of analytical grade or molecular biology grade and were purchased from commercial sources. The chemicals and reagents were stored following the manufacturers recommendations and used without further processing. Lapatinib for the animal studies was obtained from Apex Bio (#A8218). Anticoagulant Heparin for blood collection was obtained from Alfa Aesar (#A16198). For DNA fragment amplification and assembly, Q5 High Fidelity polymerase (NEB #M0491S) and the HiFi DNA Assembly Master Mix (NEB #E2621S) was used. Colony PCR was performed using the DreamTaq Green PCR Master Mix (ThermoFisher #K1081). Commercial kits for plasmid and DNA fragment isolation from PCR amplification reactions, nucleic acid gel electrophoresis and bacteria were used following the manufacturers prescribed protocol (Monarch Plasmid DNA Miniprep Kit, NEB #T1010 and the Monarch PCR & DNA Cleanup Kit, NEB #T1030). For mice plasma analysis and cytokine profiling the Proteome Profiler Mouse Cytokine Array Kit, Panel A (R&D Systems #ARY006) was used as per the manufacturers protocol. For total DNA extraction (including microbial) the tissues were processed using the Qiagen DNEasy Blood and Tissue Kit (#69504) as per the manufacturer's recommendation.

Antibodies used in the disclosed methods are summarized below in Table 1.

TABLE 1
Summary of Antibodies Employed in the Experimental Methods

| Antibody | Dilution | Application |
|---|---|---|
| TEV Cleavage Site Monoclonal Antibody (ThermoFisher #MA1- | 1:2000 | Western Blots |
| 124) | | |
| Goat anti-Mouse IgG (H + L) Cross-Adsorbed Secondary Antibody, | 1:10000 | |
| HRP (ThermoFisher #A16072) | | |
| IHC-plus Polyclonal Rabbit anti-Human ACTA2 / Smooth Muscle | 1:200 | Immunohistochemistry |
| Actin Antibody (IHC) (LSBio #LS-B4742) | | |
| Anti-Cleaved Caspase-3 antibody (Abcam #ab2302) | 1:100 | |
| anti-Ki-67 (30-9) Rabbit Monoclonal Primary Antibody (Ventana | Ready to use | |
| Medical System/Roche #790-4286) | dilutions | |
| Fibronectin Antibody (A-11) (Santacruz Biotech #sc-271098) | 1:20 | |
| Anti-Periostin antibody (Abcam #Ab83739) | 1:400 | |
| Collagen IV Monoclonal Antibody (PHM-12) (ThermoFisher # | 1:50 | |
| MA5-13255) | | |
| Hyaluronic Acid (HA) Antibody (abbexa #abx101090) | 1:200 | |
| Bioxcell In Vivo MAb anti-mouse PD-L1 (B7-H1) | — | In vivo antitumor study |

### Bacterial Strains and Cell Culture Conditions

Bacterial strains and plasmids used in the disclosed experimental methods are summarized in Table 2. For general cloning, TOP10 cells were used (One Shot TOP10 Chemically Competent E. coli, ThermoFisher #C404010) Strains were grown in LB agar (BD Difco #244520) and LB medium (BD Difco #244620) as appropriate with supplements such as antibiotics for transformant selection or IPTG for induction. Growth rates were measured via optical density measurements at 600 nm.

TABLE 2
Bacterial Strains and Plasmids Employed in the Experimental Methods

| | Genomic | |
|---|---|---|
| Strain abbreviation | modification/plasmid | Description |
| EcN | — | Escherichia coli Nissle 1917 commercially available as |
| | | Mutaflor (GenBank: CP007799.1) |
| EcN | pKM208 | EcN with λ-red recombination system (gamma, exo and |
| | | beta) (Addgene Plasmid #13077) |
| TOP10 | pBbS18c-RFP | TOPP10 strain with plasmid for preparing |
| | | chloramphenicol cassette (Addgene Plasmid #126201) |
| ΔE | Δnlpl | Hypervesiculating EcN |
| DH5α | pOGG005 | High copy number plasmid backbone (Addgene Plasmid |
| | | #113980) |
| TOP10 | pecol19A-ClyA-Hy | Plasmid cloning |
| ΔElux | pAKgfplux1 | Hypervesiculating EcN with luciferase for BLI in-vivo |
| | | (Addgene Plasmid #14083) |
| ECHy | pecol19A-ClyA-Hy | EcN expressing membrane confined Cytolysin A- |
| | | Hyaluronidase (CHy) |
| ΔECHy | pecol19A-ClyA-Hy | Hypervesiculating EcN Expressing membrane confined |
| | | Chy |

Primers used in the experimental methods are set forth in Table 3 below (5′-3′).

TABLE 3
Primers Employed in the Experimental Methods

| SEQ | | SEQ | | Use |
|---|---|---|---|---|
| ID | | ID | | |
| No: | Forward primers | NO: | Reverse primers | |
| 1 | TAGCAGCTGCAAAGAGG | 2 | CGGTACCCACGTGGGGCCC | Amplify |
| | AGAAACACCATATGACA | | GTCGACTCTAGAGGATCCT | CHy gene |
| | GAGATTGTTGCGGACAA | | CACGCAGGTGCAATTGTCGTTA | blocks |
| 3 | AGAGTCGACGGGCCCCA | 4 | ATGGTGTTTCTCCTCTTTGCAGC | Amplify |
| | CGTGGGTACCGTTTAAA | | | vector |
| | CCCCGGGTGTACAAGTA | | | backbone |
| | CTAAGCTTC | | | |
| 5 | CTATTGCTGGTCCGATTC | 6 | ATGAAGCCTTTTTTGCGCT | Ds CmpR |
| | TGCCAGGTCATCTTGGTC | | GGTGTTTCGTTGCGACAG | gene |
| | CTGGCCCAGTTACGCCCC | | CATGATCGGCACGTAAGAGGTTC | |
| | GCCCTG | | | |
| 7 | CATGTAGTACGTGTGCC | 8 | AACCTGGCCTATTTCCCTAAAG | Colony |
| | TCAA | | | PCR for |
| | | | | CmpR gene |
| 9 | AGCGAAAGAGATTTGCCGGA | 10 | ACGCCACTGCCCTTTATTGA | PCR for |
| | | | | luxA |
| | | | | fragment |
| 11 | CTTGGTGGGCGACATTAAGA | 12 | CGACCTCTGGGACTTCAAATAA | PCR for |
| | | | | pecol19A- |
| | | | | ClyA-Hy |
| | | | | fragment |
| 13 | ATAATACCGCGCCACATAGC | | — | Additional |
| | | | | Sequencing |
| 14 | TTGACAGCTAGCTCAGTCC | | — | primer for |
| 15 | ATACCAAAAGCGCCACGGTA | | — | CHy |
| | - | 16 | CCATCTAATTCAACAA | |
| | | | GAATTGGGACAAC | |

The metastatic triple negative murine breast cancer 4T1 cells (ATCC CRL-2539, passage number 5 to 15) and human MDA-MB-231 cells ((ATCC HTB-26, passage number 5 to 15) were cultured in RPMI (Gibco #21875034) containing 10% fetal bovine serum (Gibco #26140079) and 5% Penicillin-Streptomycin (Gibco #15070063). Pancreatic cancer cells-Human Panel (ATCC CRL-1469)) and mice derived RInk1 (generated as previously reported by Seeley et al.,) and MC38 colon cancer cells (Kerafast #ENH204-FP) were cultured in DMEM (Gibco #12430112) containing 10% fetal bovine serum (Gibco #26140079), 1×HEPES (Thermofisher #15630080) and 5% Penicillin-Streptomycin (Gibco #15070063). All the cells were maintained at 37° C. with 5% CO₂ in air and sub-cultured twice weekly.

### Generating Bacterial Competent Cells

Chemically competent cells were made using the TSS buffer or using the 100 mM CaCl2) solution and electrocompetent cells were made using deionized water as reported earlier (C. E. Seidman, K. Struhl, J. Sheen, T. Jessen, Curr Protoc Mol Biol 2001, Chapter 1, Unit 18; C. T. Chung, S. L. Niemela, R. H. Miller, Proc Natl Acad Sci USA 1989, 86, 2172).

### Constructing the ΔE Strain

For the nlpI gene deletion a chloramphenicol resistance cassette with ends overlapping with upstream and downstream regions of the nlpi gene was amplified (30 bp overlapping ends). The dsDNA was electroporated into EcN cells with the k-red recombinase system and the transformants were selected as per the procedure reported earlier (K. C. Murphy, K. G. Campellone, BMC Mol Biol 2003, 4, 11).

### Phenotyping ΔE for Enhanced OMV Production

After confirming deletion of the n1pI gene, ΔE was evaluated phenotypically for assessing the increase in OMV production via protein measurements with the bicinchoninic acid assay (BCA). ΔE produced a higher OMV yield, 1.75±0.2 mg/L which is approximately two times greater in comparison to the non-hypervesiculating EcN (0.85±0.31 mg/L). ΔE and its OMVs were analyzed via Transmission Electron Microscopy (TEM) and displayed nanosized vesicles that had diameters ranging from 80 to 400 nms. (FIG. 2B and FIG. 2C) The nanosized nature and size distribution of the isolated OMVs was further confirmed via dynamic light scattering (DLS), with the results displayed in FIG. 2D. The mean vesicle diameter was calculated as 101.47±25.85 nm while the polydispersity index (PDI), which is a measure of particle size distribution was between narrow to moderately polydisperse (0.2-0.3).

Constructing pecol19A-ClyA-Hy and ΔECHy

The plasmid pOGG005 was initially modified by introducing an MCS site using restriction digestion (PsiL and PacI). A fusion protein was then designed having a J23119 promoter, followed by an RBS, ClyA (Escherichia coli (strain K12) 303 amino acids, UniProtKB #P77335), TEV site and Hy (Streptomyces koganeiensis, 217 amino acids, UniProtKB #A0A0U2E2J7) and the geneblocks were obtained from IDT. Codon optimization was performed using available online tools. The geneblock and pOGG005-MCS plasmid blackbone were amplified with overlapping ends that were subsequently joined using the HiFI assembly master mix as per the manufacturer's instruction. The plasmid was cloned into TOP10 cells followed by subcloning in ΔE cells to obtain ΔECHy.

All DNA assembly fragments gene modifications were sequence verified by colony/plasmid PCR, nucleic acid gel electrophoresis (1.2% and 1.5% agarose gel) and Sanger sequencing (DNA Sequencing and Genotyping Core, Cincinnati Children's Hospital Medical Center). Plasmid designing and sequence alignments were performed using Snapgene and NCBI blast.

### OMV Isolation and Characterization

A sequential differential centrifugation protocol was developed for isolating the OMVs. In brief, bacteria were grown overnight until they reached the stationary phase and 1 ml of this culture was used to inoculate 1 L of LB Broth. The bacteria were grown until they reached an OD of approximately 1.5 units. The culture was cleared of bacteria by centrifugation (8000 g, 4° C., 15 minutes) followed by concentrating the supernatant using Pierce protein concentrators (30 KdA, ThermoFisher #88531). 70 ml of the concentrated supernatant was then ultracentrifuged at 91000 g for 4 hours. The pellet obtained was resuspended in PBS pH 7.4, followed by washing with Amicon Ultra-0.5 Centrifugal Filter Unit (Millipore #UFC500308) filters to remove the broth completely. The resulting dispersion was sterile filtered through 0.22 μm PVDF syringe filters (Cole-Parmer #UX-06060-62). OMVs were then characterized for size and size distribution using Dynamic Light Scattering (Zetasizer Nano ZS, Malvern Instruments) and electron microscopy (Hitachi H-7650 Transmission Electron Microscope). For long term storage, the OMVs were stored at −80° C. for further processing downstream.

### SDS-PAGE Western Blot

Bacterial lysates were prepared using the Bacterial Protein Extraction Reagent (ThermoFisher #89821) following the manufacturer instruction. Protease and phosphate inhibitors were added as per the general requirements of preparing lysates for western blots. Bacterial and OMV lysates were separated using 8% Bis-Tris precast gels (Thermofisher #NW00080BOX) followed by semi dry transfer onto a 0.45 μm nitrocellulose membrane. The membranes were blocked with 5% BSA in PBST followed by incubation with primary and secondary antibodies. Chemiluminescent substrate was added (Thermofisher #34579) and detected using the Biorad ChemiDoc imaging system.

### Hyaluronidase Assay

HA agarose plate method: The assay method was performed as reported earlier (D. Grenier, J. Michaud, J Clin Microbiol 1993, 31, 1913). In brief, a 1% agarose solution (20 ml) containing 0.4 μg/ml HA and 1% BSA fraction IV was poured onto sterile 30 mm petri dishes. 100 μl droplet of bacterial strain containing 10\hat{ }6 bacteria was dropped onto the plate followed by air drying and overnight incubation at 37° C. The plates were flooded with 2N glacial acetic acid followed by imaging for transparent and opaque regions for determining the zones of HA degradation.

HA degradation turbidimetric assay: The assay method was performed according to standard procedures. (Y. Lou, P. C. McDonald, A. Oloumi, S. Chia, C. Ostlund, A. Ahmadi, A. Kyle, U. Auf dem Keller, S. Leung, D. Huntsman, B. Clarke, B. W. Sutherland, D. Waterhouse, M. Bally, C. Roskelley, C. M. Overall, A. Minchinton, F. Pacchiano, F. Carta, A. Scozzafava, N. Touisni, J. Y. Winum, C. T. Supuran, S. Dedhar, Cancer Res 2011, 71, 3364). The CTAB reagent was prepared by dissolving cetyltrimethylammonium bromide at 2.5% w/v in 100 ml of 2% w/v NaOH solution. Overnight cultures of bacteria containing different concentration of HA (0.1, 0.2 and 0.4 μg/ml) were cleared of the bacteria and 50 μl of each sample was mixed with 0.1 M phosphate buffer pH 7 in a 96 well microplate. The plates were incubated at 37° C. for 15 minutes followed by the addition of 100 μl of CTAB reagent. The plates were incubated for 10 min at 37° C. followed by measuring optical density at 600 nm in triplicates.

### Animal Models

6-8 weeks old female BALB/cJ and male C57BL/6 mice (Jackson Labs) were used in all experiments. For tumor induction, 106 tumor cells/0.1 ml saline (4T1, MDA-MB-231, RInk1, PANC1, MC38) were injected subcutaneously in the shaved right flank of anesthetized (2% isoflurane) mice. Tumor measurements were taken every alternate day and tumor volume was calculated using the following formula: 0.5*length*width*width. For therapeutic assessment or bioluminescent imaging, each bacterial dose contained 106 bacteria suspended in 0.1 ml saline was administered intravenously. Lapatinib was suspended in a solution containing 0.5% w/v HPMC and 0.10% w/v Tween 80 and each dose delivered 5 mg/Kg orally. At the study end point the mice were euthanized via carbon dioxide inhalation and cervical dislocation followed by collection of organs. For cytokine profiling, blood withdrawal was performed after a period of 24 hours via the submandibular vein and collected in tubes containing heparin as an anticoagulant (40 U/ml of blood). Collected blood was immediately centrifuged at 2000 g, 4° C. for 15 minutes to obtain the plasma as supernatant. Cytokine profiling was conducted using a membrane-based sandwich immunoassay kit as per the manufacturer's instructions.

### Bioluminescence Imaging

Bioluminescence images were acquired for 1-2 mins using the Perkin Elmer IVIS Spectrum In-Vivo Imaging System for quantification of radiance of the bioluminescent signals from the regions of interest.

### PET Imaging of OMVs In Vivo

For PET imaging of OMVs, a 64Cu-Yb complex was prepared as previously reported (N. A. Siddiqui, H. A. Houson, N. S. Kamble, J. R. Blanco, R. E. O'Donnell, D. J. Hassett, S. E. Lapi, N. Kotagiri, JCI Insight 2021, 6). OMVs were incubated with 1 mCi of the radioactive complex for 1 hour at 37° C. Size exclusion chromatography was used to remove the free complexes and purify OMVs (PD-10 columns, Cytiva Life Sciences #17085101). Vesicles were concentrated using Pierce Protein Concentrators (30 kDa, Thermo Scientific #88531) before administration in mice (BalbC with 4T1 tumors ≈200 mm3, n=3). 4 hours post intravenous administration, small animal PET scan was performed on a μPET scanner (Siemens Inveon). Mice in the supine position under anesthesia were placed on the imaging gantry with continued warming. For anatomical reference overlay, a CT scan was performed (80 kVp, 500 μA, at 120 projections) followed by the acquisition of PET images for 15 minutes with real-time reconstruction. Spatial resolution was determined by ordered subset expectation maximization in 2D. Histogramming and reconstruction were applied using the system onboard Inveon software. Post-processing was carried out using the Inveon Research Workplace software.

### Immunohistochemistry

Tumor tissue was isolated and fixed with 10% v/v formalin followed by replacement of storage solution with 70% v/v ethanol. Immunohistochemistry slides were prepared via paraffin processing and were developed by the Pathology Research Core at the Cincinnati Children's Hospital Medical Center. The slides were imaged under ×100 and ×400 magnification using the Leica DMi8 Widefield Fluorescence/Brightfield Microscope. The images were quantified with ImageJ.

### Statistical Analysis

All statistical analyses were performed using the GraphPad Prism 8 software. The survival cure was analyzed using the nonparametric Log-rank (Mantel-Cox) test. Ordinary two-way ANOVA and paired t-test (Wilcoxon) was used to compare means between different groups. Values of p<0.05 were considered to be statistically significant.

### Example 2. Genomic Deletion on nlpI from EcN to Obtain Hypervesiculating ΔE

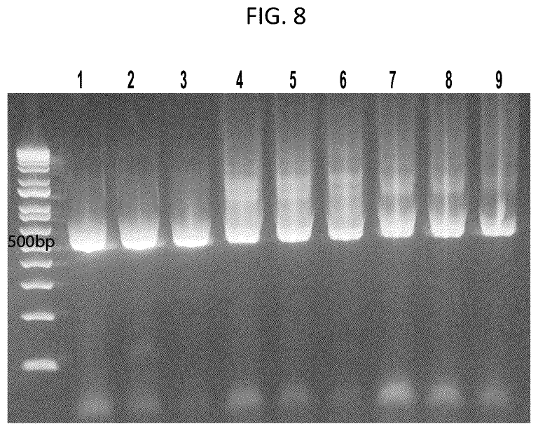

To develop a hypervesiculating strain of EcN, its genome was modified by deleting the nlpI gene as reported previously for similar strains of Escherichia coli. (D. J. Chen, N. Osterrieder, S. M. Metzger, E. Buckles, A. M. Doody, M. P. DeLisa, D. Putnam, Proc Natl Acad Sci USA 2010, 107, 3099). Deletion of the nlpI gene has been reported to be safe for bacterial membrane integrity and stability. EcN expressing the k-red recombination system proteins, gamma, exo and beta, was electroporated with a double stranded DNA encoding the chloramphenicol resistance cassette gene with overhangs homologous to the gene sequences upstream and downstream of the nlpI gene. The k-red recombineering system excises the nlpI gene from the genomic DNA and replaces it with the electroporated dsDNA containing the chloramphenicol cassette (FIG. 2A). Bacterial colonies obtained after chloramphenicol based antibiotic plate selection were analyzed via colony polymerase chain reaction (PCR) (FIG. 8) to confirm the integration of the chloramphenicol cassette. The PCR amplified product was further analyzed by Sanger sequencing which reconfirmed the previous results. Thus, the nlpI gene was successfully replaced to obtain a hypervesiculating strain of EcN, ΔE.

### Example 3. Phenotyping ΔE for Enhanced OMV Production

ΔE was evaluated phenotypically for assessing the increase in OMV production via protein measurements with the bicinchoninic acid assay (BCA). ΔE produced a higher OMV yield, 1.75±0.2 mg/L which is approximately twice in comparison to the non-hypervesiculating EcN (0.85±0.31 mg/L). ΔE and its OMVs were analyzed via Transmission Electron Microscopy (TEM) and observed nanosized vesicles that had diameters ranging from 80 to 400 nm respectively, in agreement with the size range reported elsewhere (FIGS. 2B, 2C). The nanosized nature and size distribution of the isolated OMVs was further confirmed via dynamic light scattering (DLS) (FIG. 2D). The mean vesicle diameter was calculated as 101.47±25.85 nm while the polydispersity index (PDI), which is a measure of particle size distribution was between narrow to moderately polydisperse (0.2-0.3). The production of OMVs is a natural biological process inherently controlled by its mechanism of formation and release (blebbing of the outer membrane and explosive cell lysis). Thus, a variation in size distribution and Z-average is reasonably expected with each batch isolated.

### Example 4. Designing Cytolysin a (ClyA)-Hyaluronidase (Hy) (CHy) Fusion Protein

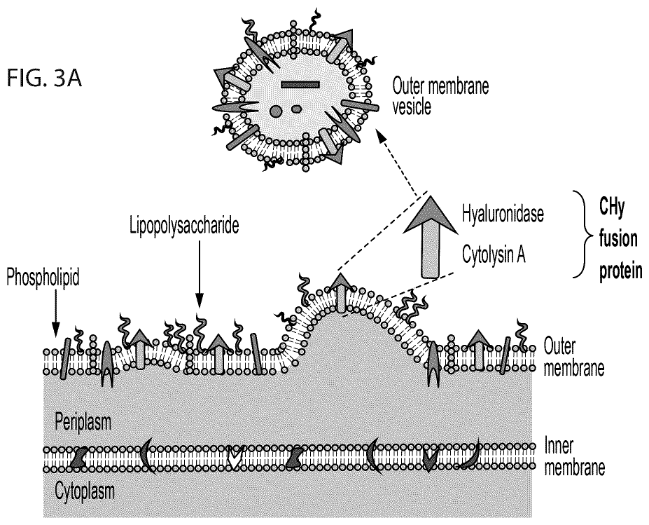

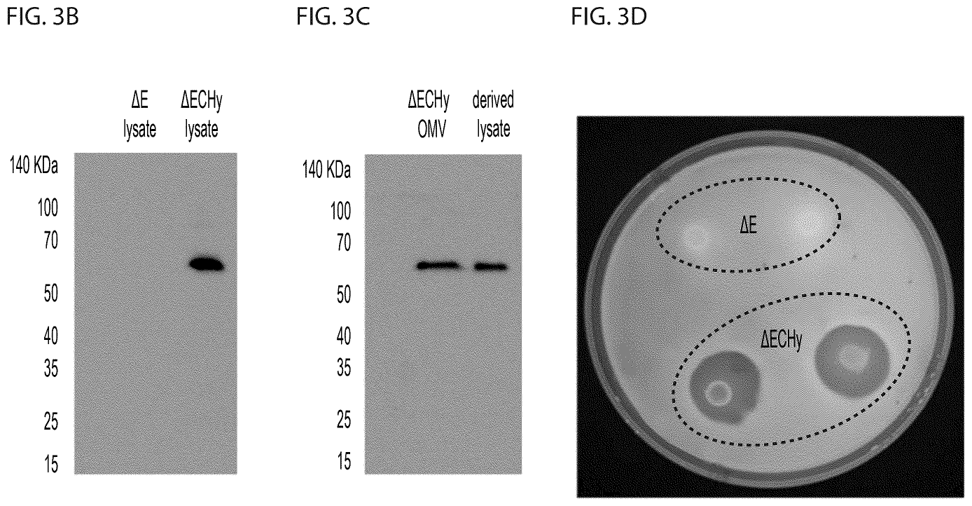

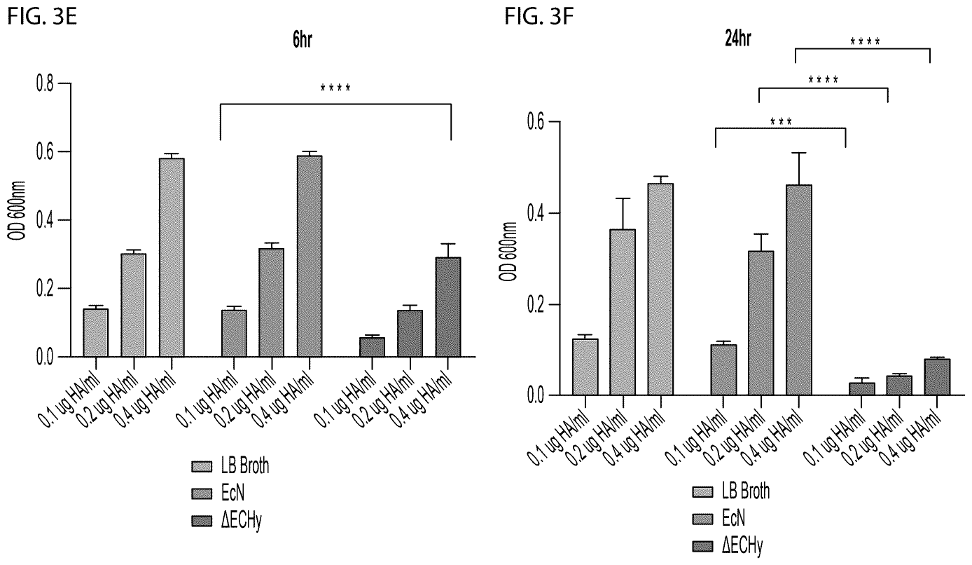

Previously, human and bovine forms of Hy such as the sperm surface protein PH-20 (PH20/SPAM 1, 509 amino acids) have been cloned for therapeutic objectives. For complete catalytic activity, the enzyme needs to undergo post-translational modifications or else the enzyme would perform sub-optimally, especially when being cloned in bacteria. Hence, an alternate form of Hy of bacterial origin was selected that does not require post-translational modifications. A 217 amino acid sequence of Hy from Streptomyces koganeiensis (UniProtKB #A0A0U2E2J7) has been reported to be enzymatically active against high molecular weight HA which was subsequently selected for cloning and expression. Moreover, its small size (˜24.5 kDa) makes it a favorable candidate for synthetic cloning. Further, the enzyme needs to be translocated onto the bacterial outer membrane which would then be subsequently packaged inside the OMVs as they are being formed. Fusion of proteins at the C terminal of ClyA has been extensively studied for bacterial surface and OMV localization. ClyA is 34 kDa transmembrane pore-forming hemolytic protein (UniProtKB #P77335) known to be cytolytic towards mammalian cells and macrophages. Fusion of fluorescent proteins and enzymes with the ClyA gene has been utilized to transport and localize these protein onto the bacterial membrane and further onto the OMVs without any loss in functional activity (FIG. 3A). ClyA retains its pore forming cytolytic activity even after the addition of peptide fragments on either ends as shown previously. Thus, ClyA serves two roles—primarily as an anchoring protein for Hy and secondarily to exert a cytotoxic effect against cancer cells. Hence, for bacterial membrane localization a fusion protein was designed, CHy encoding ClyA on the 5′ end and Hy on the 3′ end with a TEV cleavage site in the middle having glycine (G₄) spacers/linkers on either side (≈56.5 kDa). The fusion protein has a sequence according to SEQ ID NO: 17.

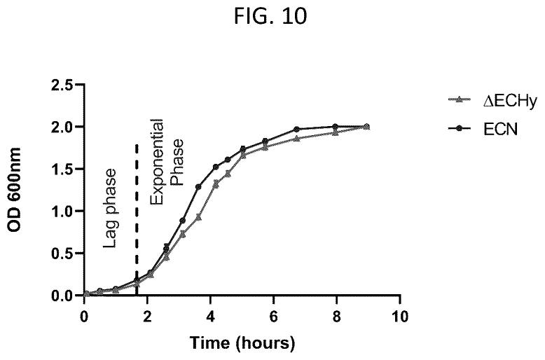

EcN codon optimized gene blocks were obtained for the fusion protein and incorporated it into a high copy number plasmid to obtain pecol19A-ClyA-Hy (FIG. 9). The designed plasmid has a strong E. coli constitutive promoter, J23119 that would drive the production of the fusion protein without an inducer. The assembled plasmid was sequence verified and subcloned into ΔE to obtain ΔECHy. Further, the metabolic burden due to the production of recombinant proteins can also affect the growth of bacteria. The J23119 promoter and ClyA mediated membrane localization have been reported to have negligible effects on bacterial hosts cellular capacity and growth rate. The effect of the plasmid on the growth rate of ΔECHy was analyzed and found to be comparable with ECN (FIG. 10).

### Example 5. Localization of Functional CHy in ΔECHy OMVs

Next, ΔECHy and its derived OMVs were analyzed for the presence of recombinant CHy. ΔECHy lysates and its OMVs were tested by SDS-PAGE immunoblotting with antibodies specific for the TEV cleavage site. Bands at the molar mass corresponding to 55.6 kDa were observed in both samples which were absent in the negative control (i.e., ΔE lysate) (FIGS. 3B, 3C). This confirmed that ClyA is an efficient carrier protein for translocation of fused Hy on OMVs. CHy was evaluated for its functionality via a qualitative agarose plate-based HA degradation assay. The plate-based method is a test to analyze HA degradation by observing zones of degradation/clearance around regions of bacterial growth in response to precipitation of HA. Clear regions around the circular bacterial disk of ΔECHy were observed after 24 hours which were transparent in comparison to the region inoculated with ΔE, which was opaque (FIG. 3D). A quantitative assay was also performed for analyzing Hy mediated HA degradation using turbidimetry. This spectrophotometric assay is based on the precipitation of solubilized HA using a surfactant, cetyltrimethylammonium bromide (CTAB) followed by measuring the optical density at 600 nm. Three different concentrations of HA 0.1, 0.2 and 0.4 μg/ml were analyzed for three different groups, namely LB broth, ΔE and ΔECHy. After a period of 6 hours, ΔECHy showed an approximate 50% reduction in optical density for all the concentrations tested, which increased to 80% after a period of 24 hours. Insignificant changes in optical density were observed for the LB Broth negative control and the ΔE groups (FIGS. 3E, 3F). The assays confirmed the successful synthesis of a hypervesiculating strain of EcN (ΔECHy) that is ready for in-vivo targeting of tumors and delivering recombinant Hy and ClyA, capable of degrading HA and killing tumor cells, respectively.

### Example 6. Tumor Screening for the Presence of Fibrotic Elements

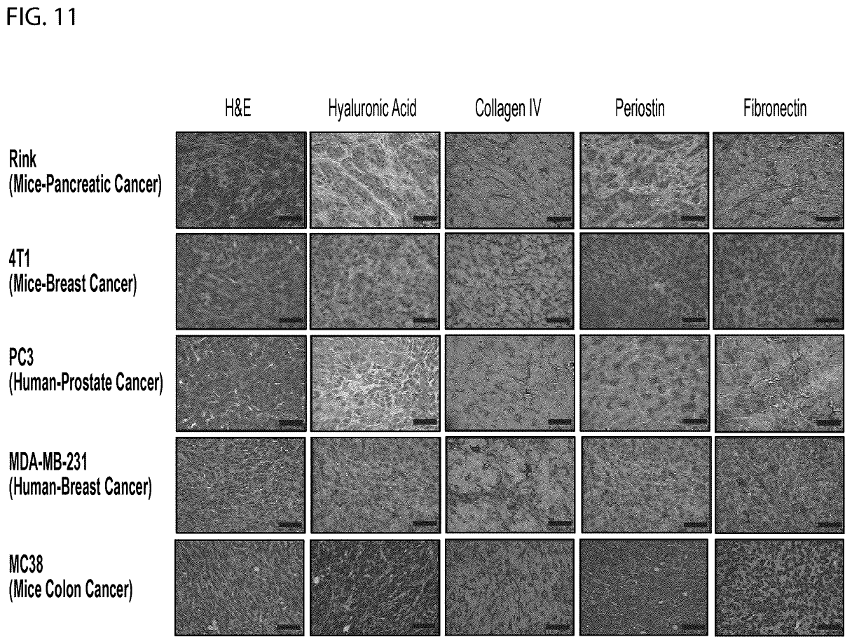

Multiple tumor models, both xenograft and syngeneic, in immunocompromised and immunocompetent mice were screened for evaluating the composition of extracellular matrix proteins such as HA, periostin, fibronectin and collagen IV. Tumor specimens of human origin breast (MDA-MB-231) and prostate (PC3) cancers; and murine origin breast (4T1), colon (MC38) and pancreatic (RInk1) cancers were analyzed for the fibrotic markers by immunohistochemistry (IHC), where MDA-MB-231, 4T1, MC38 and RInk1 tumors were shown to contain significantly higher amounts of these fibrotic elements (FIG. 11) over PC3 tumors. Targeting one or a combination of these elements for stromal reengineering is expected to improve the perfusion dynamics in tumors and thereby have significant improvement in drug delivery of small molecules and macromolecules.

### Example 7. Localization of ΔE and OMVs in Tumor Models

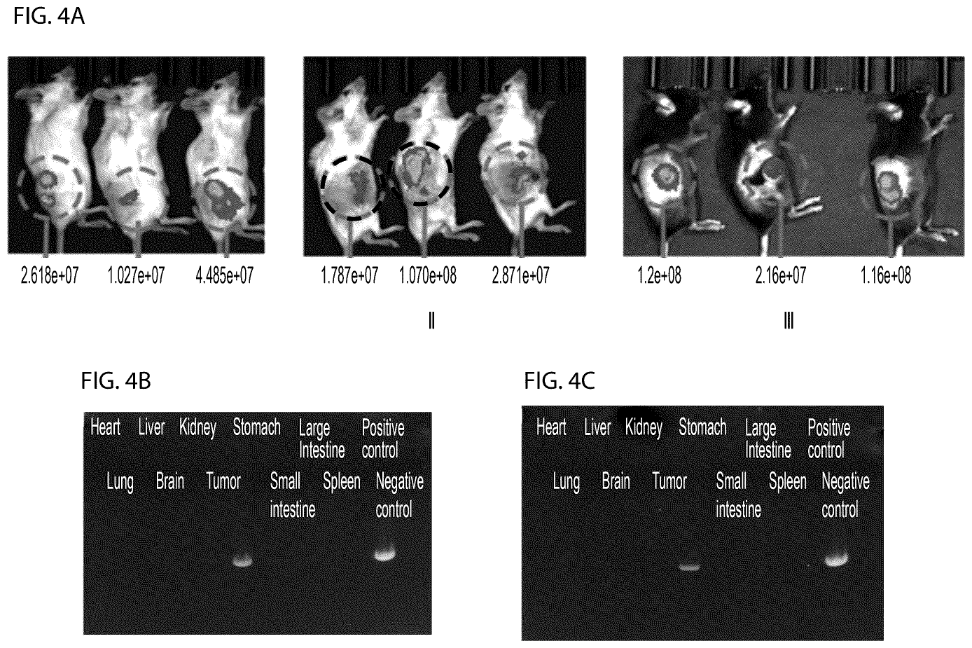

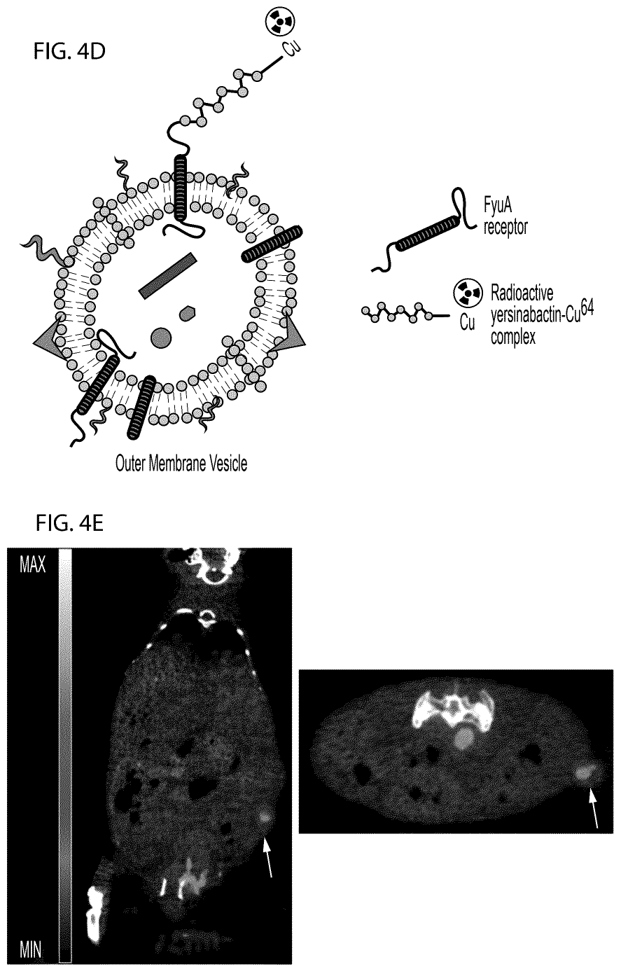

4T1 (breast) and MC38 (colon) were selected as tumor models for further evaluation of ΔECHy due to the syngeneic nature of these models, consistent growth kinetics, and the reliable development of a densely fibrotic and hypoxic tumor that is essential to study the localization of engineered facultative anaerobes. ΔElux, a bioluminescent strain containing the constitutively expressed luciferase (lux) reporter gene, was administered intravenously in 4T1/BALB/cJ and MC38/C57BL/6 tumor bearing mice once a tumor size of 200-300 mm³ was attained. After allowing 24 h for tumor seeding and populating, consistent bioluminescence (BLI) signals were observed to originate from the tumors in both the models thereby indicating that the engineered EcN, i.e., ΔElux, retains the tumor homing properties (FIG. 4A). The BLI signals were exclusively originating from tumors suggesting successful seeding and proliferation of the bacteria in the immunocompromised TME and the clearance of bacteria from all major organs. While BLI is a highly specific modality to study growth kinetics of living therapeutics non-invasively in vivo, it is not the most sensitive technique to accurately estimate the biodistribution profile and trace the molecular signatures of these agents in vivo. Hence, the study was repeated with an intravenous administration of ΔECHy in 4T1 and MC38 tumor models. The mice were euthanized, and various organs were collected 72 hours post administration, processed and the total DNA was analyzed for the presence of pecol19A-ClyA-Hy plasmid via PCR, a highly specific and sensitive modality, albeit invasively. The plasmid sequence was detected exclusively from tumors in both models with no signal from other organs (FIGS. 4B, 4C). These results indicate the proficiency of bacteria in selectively populating the tumor matrix in a spatial and temporal fashion with continued propagation and maintenance of their population in the hypoxic and immunocompromised tumor microenvironment.

While OMVs are generated in situ in our strategy, we sought to evaluate the biodistribution profile of OMVs to determine whether they exhibit higher tropism to other organs, over tumor, in the event there is infiltration of OMVs into the systemic circulation from the tumor. OMVs akin to nanoparticle-based carriers should exhibit tumor-tropism, accumulation and penetration characteristics via the enhanced permeability and retention (EPR) effect. To test this hypothesis and simulate biodistribution of OMVs from the systemic vasculature, OMVs were isolated from the hypervesiculating bacteria and purified before radiolabeling them with copper-64 labeled Yersiniabactin (⁶⁴Cu-YbT), a radioactive siderophore complex, to track them in vivo using Positron Emission Tomography (PET) imaging. ⁶⁴Cu-YbT complex binds selectively to the outer membrane FyuA receptor, a siderophore transporter, expressed in multiple species of Escherichia coli and its OMVs, including Nissle and has been shown to play a role in bacterial copper acquisition and import. Hence, the radioactive complex was used to tag OMVs for tracking them in-vivo (FIG. 4D). Radiolabeling OMVs with ⁶⁴Cu-YbT and processing to remove the unbound complex yielded approximately 7.4 MBq of radioactivity in 3 mg of OMVs. A 3-dimensional PET/CT imaging session was performed 4 hours post intravenous administration of 300 μg of radiolabeled OMVs (0.74 MBq). Strong signals were observed originating from the tumor location, compared to other major organs (FIG. 4E). This result illustrates the high tumor-tropism of OMVs and their low dissemination potential beyond the tumor site. Thus, a hypervesiculating strain of EcN localizing at different sites within tumors and releasing OMVs are likely to be retained within the tumor matrix due to the EPR effect.

### Example 8. ΔECHy-Mediated Tumor Stromal Remodeling and Potentiation of Lapatinib Response in 4T1 Syngeneic Tumor Model

After confirming the tumor homing property of ΔE and demonstrating the expression of functional CHy on the bacterial surface and in the OMVs, the next goal was to evaluate the efficiency of HA degradation and its effect on improving chemotherapeutic outcomes in vivo. It is advantageous to evaluate ΔECHy in syngeneic models since it would allow testing the live biotherapeutic in immunocompetent mice and take advantage of the native immune responses triggered against the tumor in combination with synergistic therapeutic agents such as EGFR TKIs. There are several studies that implicate HA in TKI treatment resistance across several tumor types. For example, HA has been shown to be associated with modifying responses to TKIs by activating the CD44 and the cell-surface RHAMM receptors that also act as co-receptors for activating transmembrane tyrosine kinases (e.g., EGFR, c-MET, PDGFR and ERK) and their multiple pathways downstream (e.g., Rho GTPases and Ras GTPases in the Rho and MAPK signaling pathways). Stromal HA secreted by CAFs is shown to be essential in developing lapatinib resistance by protecting against the accumulation of drug and the resulting pro-survival CAF signaling pathways such as the JAK2/STAT3 pathway and improving the apoptotic threshold of cancer cells. Intratumoral injection with Hy has been found to significantly retard tumor progression and enhance the sensitivity of cancer cells towards lapatinib. Thus, for a small molecule like lapatinib significant improvements would be expected in chemotherapeutic outcomes due to enhanced tumor penetration and the lowered tendency of tumor cells to develop resistance mechanisms as a result of the reduced stromal HA.

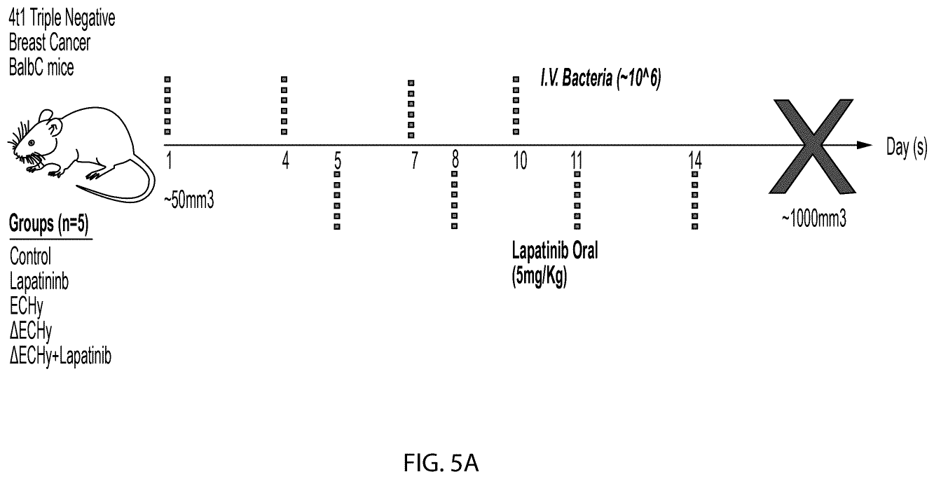

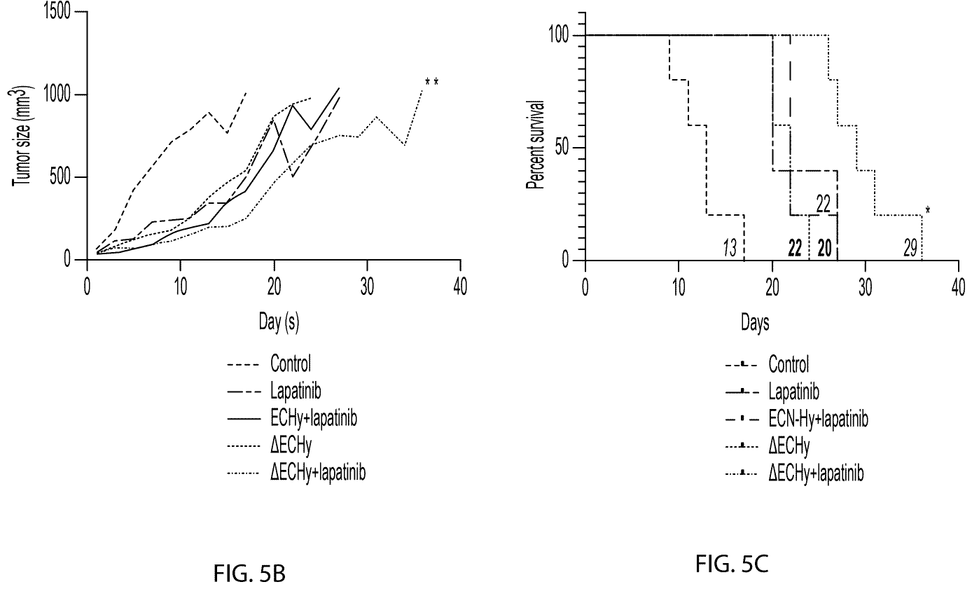

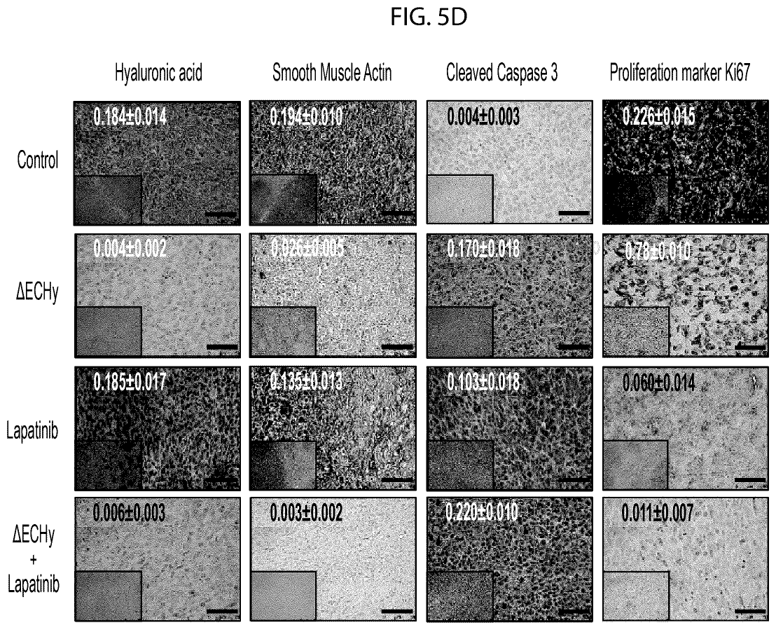

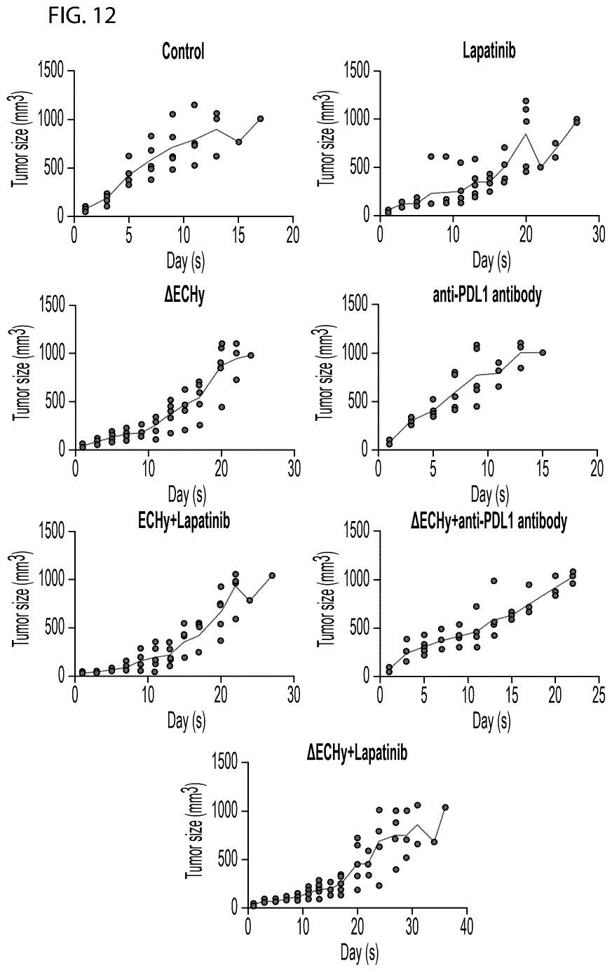

Lapatinib is a selective small-molecule dual-TKI of HER2 and EGFR which has been shown to have a therapeutic effect in 4T1 tumors at multiple doses ranging from 75-100 mg/kg orally, however with HA linked CD44 signaling, development of resistance has also been reported for the drug. Thus, HA degradation should significantly enhance the activity of lapatinib and a therapeutic response would be observed at subcytotoxic doses of the drug. ΔECHy-mediated degradation of HA and the OMVs providing greater spatial penetration into the tough tumor matrix, would modulate the tumor matrix allowing the infiltration of anticancer agents such as lapatinib to deter cancer cell proliferation and modify biological mechanisms mediated by HA-CD44 signaling thereby enhancing the blockade of EGFR signaling and inhibiting the development of drug resistance. To test the hypothesis, mice were treated orally with lapatinib at a dose of 5 mg/kg (FIG. 5A). The following 5 treatment groups were assessed in parallel: control mice with saline injections, non-hypervesiculating ECHy+lapatinib, ΔECHy, lapatinib and ΔECHy+lapatinib (n=5). Tumor volume was measured every alternate day. FIG. 5B represents a line plot showing the mean tumor volume progression with time (tumor volume-time plot for each mouse for all the groups can be found in FIG. 12). 4T1 tumors grow at a fast rate and as anticipated, mice in the control group (saline) reached their experimental endpoint (tumor size of 1000 mm³) quickly with a median survival of 13 days. The tumor growth rate was found to be comparatively slower in the lapatinib treated group, with a median survival of 20 days, which would indicate that even subcytotoxic doses administered were able to attenuate tumor progression. The non-hypervesiculating ECHy and lapatinib combination showed a profile similar to the lapatinib only treatment group with a median survival of 22 days, indicating that a non-hypervesiculating strain with CHy is not able to substantially amplify and complement the therapeutic action of lapatinib. ΔECHy alone however demonstrated a progression profile similar to the two treated control groups, with a median survival of 22 days, indicating that hypervesiculation and OMV mediated ClyA distribution is causing cytolytic activity on tumor cells and concurrently improving tumor penetration of immune cells and potentiating the immune response from Hy activity. The combination of lapatinib with ΔECHy demonstrated the slowest progression in tumor growth in comparison to the other four groups tested, with a significant increase in median survival to up to 29 days (ΔECHy+lapatinib vs control: **p=0.0021, ΔECHy+lapatinib vs lapatinib: *p=0.0421, ΔECHy+lapatinib vs ECHy+lapatinib: *p=0.0173 and ΔECHy+lapatinib vs ΔECHy; **p=0.0019) (FIG. 5C). OMVs accumulate in the tumor matrix and hence hypervesiculation would lead to increased production of OMVs with CHy which would allow enhanced distribution and penetration inside the tumor matrix. Possibly, this would lead to vital differences in tumor growth and survival, even if the bacteria are themselves physically immobile and are stuck in the dense stroma, thereby improving drug penetration and action via extensive HA degradation. Hence, OMVs generated in situ distribute Hy more effectively and uniformly inside the tumors, permitting the penetration of drugs and potentiating their effect. This is likely the reason that even a sub-cytotoxic dose of lapatinib was effective therapeutically.

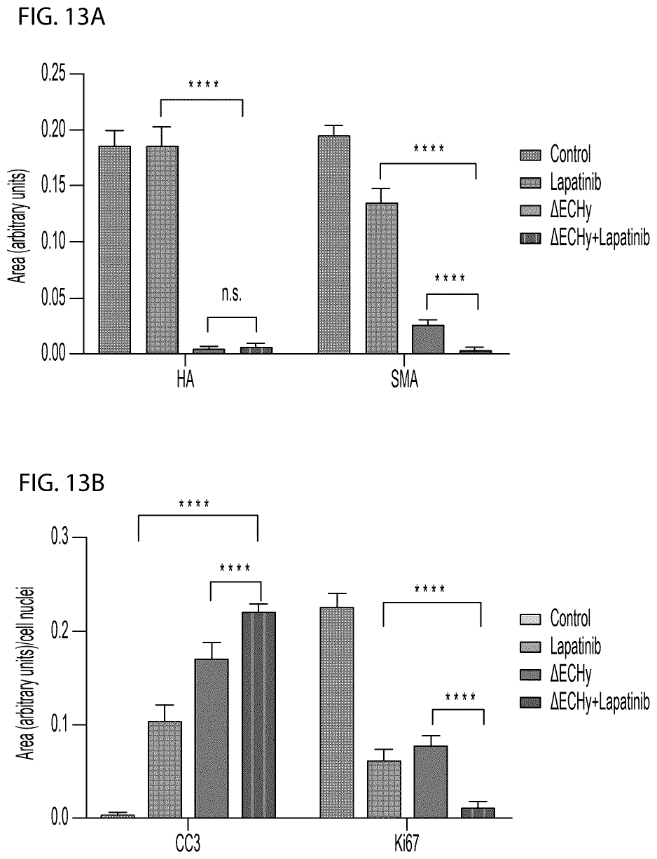

To further elucidate the stromal changes in the different treatment groups, tumor samples were analysed using IHC (FIG. 5D, 13A, 13B). ΔECHy treated tumors showed a significant reduction in extracellular HA compared to control tumors. These findings at the tissue level correlate well with the results from the tumor progression study, where it was observed that ΔECHy treated tumors facilitated enhanced therapeutic response from lapatinib in comparison to the controls. Previous studies have shown that the downregulation of CAFs population in tumors is associated with lowering of smooth muscle actin as a consequence of tumor architecture remodeling. Thus, the tumors were analyzed for smooth muscle actin as a CAFs marker. ΔECHy and lapatinib treated groups showed a higher reduction of smooth muscle actin, which suggests there is a proportional decrease in CAF numbers. Analyzing cellular proliferation is another aspect to qualitatively measure treatment response. As observed in FIG. 5D, apart from the control group, all the other treatment groups showed cells with high degree of cleaved caspase 3 (CC3) staining, which is an indicator of cells undergoing apoptosis. Further, the highest reduction in the number of proliferating cells (Ki67+) was also observed in the ΔECHy+lapatinib treatment group. These observations further support the impact of ΔECHy in enhancing tumor tissue remodeling and improving the penetration of the chemotherapeutic agent, lapatinib. Quantitative estimation of the stromal markers also indicates an ΔECHy mediated enhancement in the therapeutic effect offered by lapatinib (FIGS. 13A, 13B). Further, the reduction of HA-CD44 mediated signaling might also be a probable mechanism that lowers the probability of resistance development and enhances the EGFR blocking potential of lapatinib. These observations evidence the multipronged impact of ΔECHy as an effective agent for stromal remodeling, cytolytic therapy, and improving the penetration and activity of anticancer chemotherapeutics.

### Example 9. ΔECHy Mediated Tumor Stromal Reengineering and Potentiation of Anti-PDL1 Antibody Response in 4T1 and MC38 Syngeneic Tumor Models

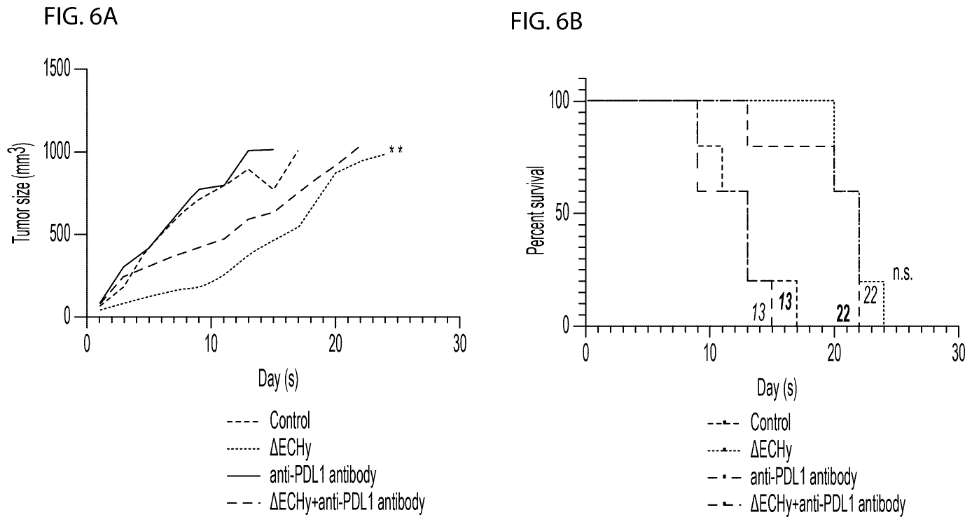

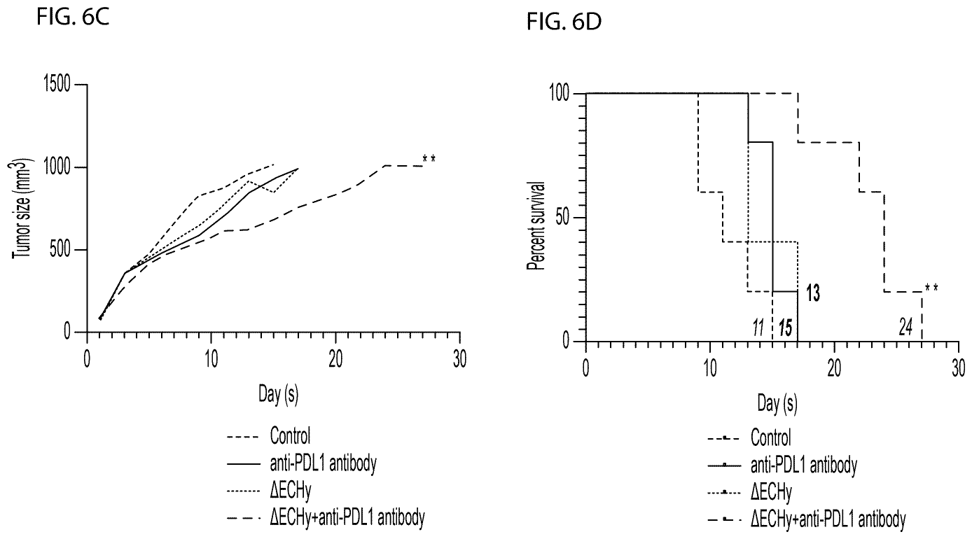

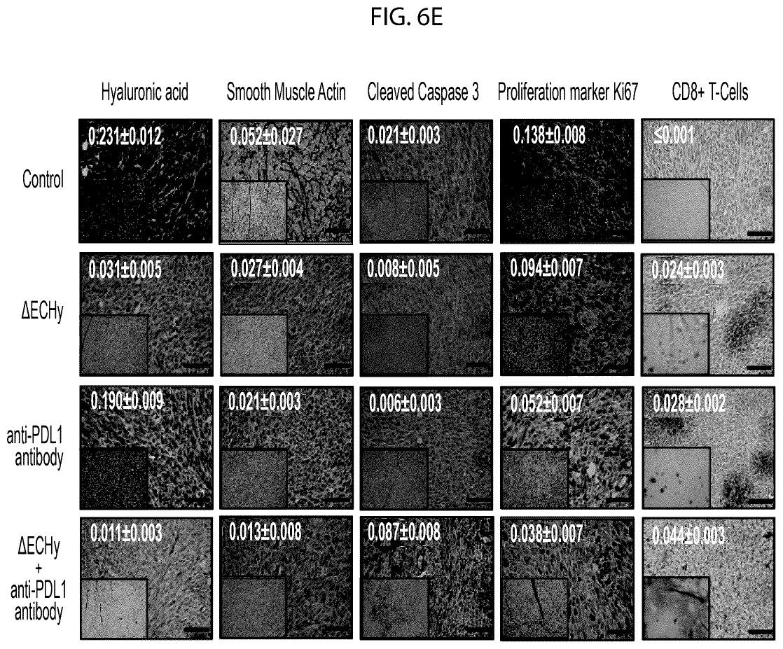

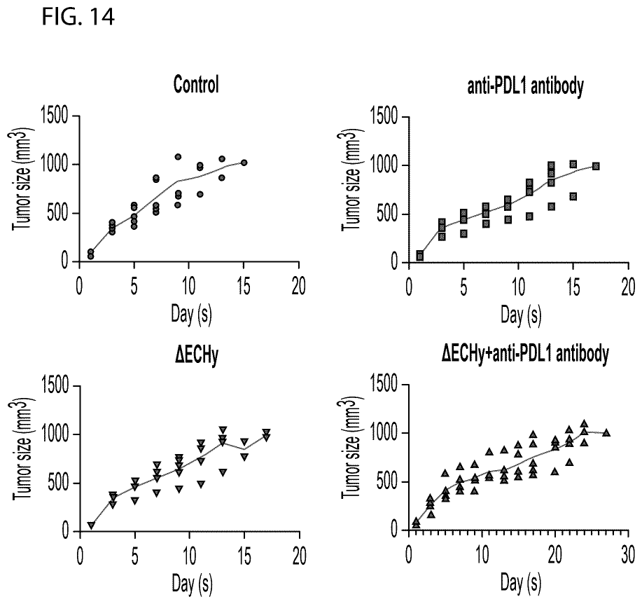

The buildup of rigid stroma in the tumor matrix is a physical barrier to macromolecules that reduces diffusion of therapeutic antibodies and immunotherapy agents to cancer cells. Hence, stromal modulation is key to enhancing the tumor penetration of macromolecules and previous studies have demonstrated that systemic administration of recombinant hyaluronidase (PEGPH20) improves the tumor accumulation of antibodies such as rituximab, trastuzumab and PDL1 antibody. Hy administration has also been shown to induce an immune response against the tumor tissue which could be a direct effect of enhanced infiltration of immune cells. Thus, to test this hypothesis, 4T1 tumors and MC38 tumors were treated with ΔECHy and immune checkpoint blockade anti-PDL1 antibodies. A subtherapeutic dose of anti-PDL1 antibody was used to resolve the therapeutic potentiation afforded by ΔECHy. Anti-PD-L1 antibody, similar to lapatinib, is tested at a subtherapeutic dose of 5 mg/kg instead of the reported dose of 10 mg/kg. In the 4T1 model, the tumor progression for the anti-PDL1 group was similar to the control group, both showing a median survival of 13 days. PD-L1 combination therapy with ΔECHy did show a significant regression in tumor growth rate in comparison to anti-PDL1 antibody treatment (**p=0.0078). However this did not translate to an improved survival outcome over what was observed with ΔECHy alone, both with a median survival of 22 days (ΔECHy+anti-PDL1 antibody vs control: *p=0.0112, ΔECHy+anti-PDL1 antibody vs anti-PDL1 antibody: *p=0.0110 and ΔECHy+anti-PDL1 antibody vs ΔECHy; not significant p=0.5164) (FIGS. 6A, 6B, 14). 4T1 tumors are typically refractory to immune checkpoint blockade, a probable reason for which could be the vast number of immunosuppressive cells such as the regulatory T-cells and the myeloid derived suppressor cells populating the tumor matrix and lesser number of CD8+ T cells and natural killer (NK) cells infiltrating the tumor. Thus, a possible reason for such an outcome could be the immunosuppressive nature of tumor or the low dose being insufficient to elicit a response.

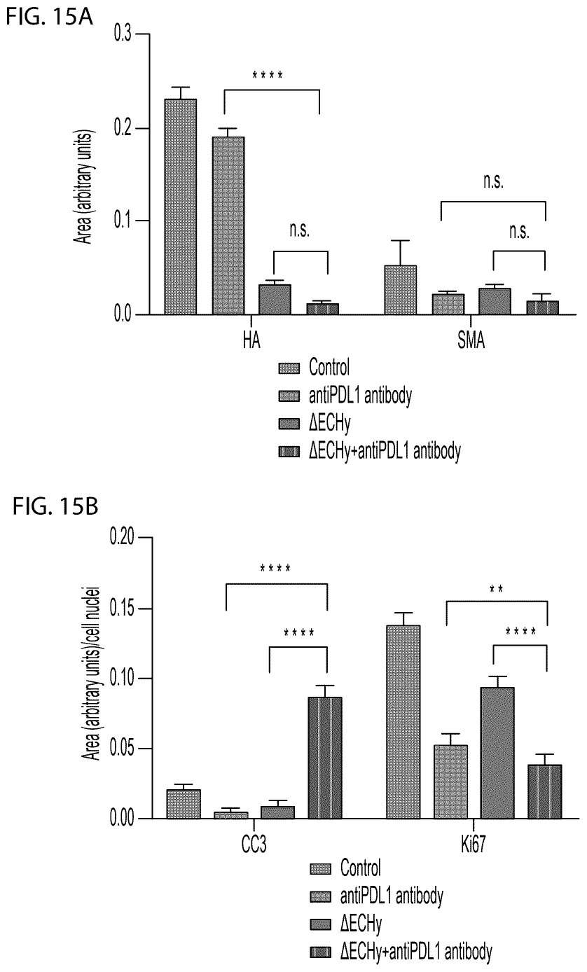

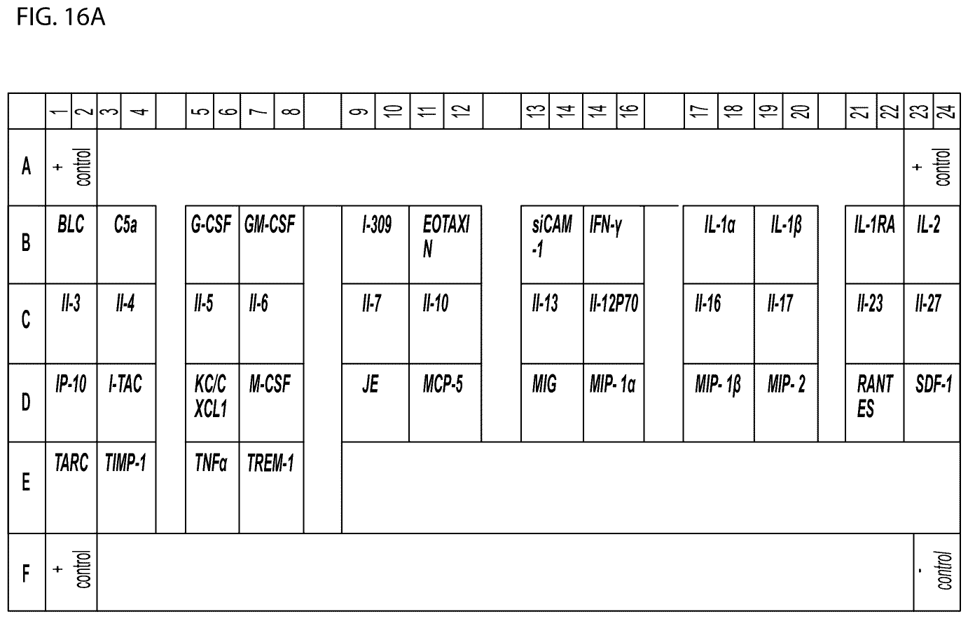

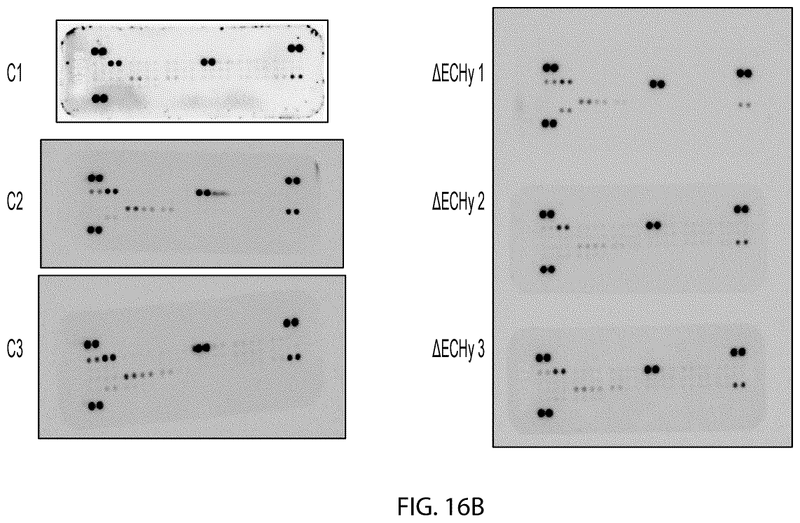

In MC38 tumors, ΔECHy and anti-PDL1 groups showed a slight reduction in the tumor growth rate in comparison to control (saline) with a modest increase in survival from 11 days (control) to 15 (anti-PDL1) and 13 (ΔECHy) days, respectively. However, the ΔECHy and anti-PDL1 combination groups displayed a definite improvement in tumor survival with the median survival to up to 24 days (ΔECHy+anti-PDL1 antibody vs control: **p=0.0017, ΔECHy+anti-PDL1 antibody vs anti-PDL1 antibody: **p=0.0052 and ΔECHy+anti-PDL1 antibody vs ΔECHy; **p=0.080) (FIGS. 6C, 6D, 14). Further, different treatment groups were analyzed by IHC (FIGS. 6E, 15A, 15B). Similar to the results in the lapatinib study, HA synthesis was again found to be qualitatively reduced in the ΔECHy treated groups. Smooth muscle actin was also found to be reduced in all the treatments groups in comparison to the untreated control thereby indicating positive stromal reengineering. The combination therapy of ΔECHy with the immune checkpoint inhibitor antibody showed a qualitative and quantitative reduction in cellular proliferation. Analysis of the tumor tissue for the cytotoxic T-cell marker, CD8⁺, and the apoptosis marker, CC3, showed a widespread tissue distribution of cytotoxic T-cells and increased cellular apoptosis. In comparison to the other groups, stromal engineering clearly potentiated the antibody therapy by enhanced permeation of immune cells and the therapeutic antibody. In comparison to 4T1 tumors, MC38 showed a definite improvement in survival with the combination of ΔECHy+PD-L1 antibody. MC38 tumors exhibit a vast expansion of T-cell populations and macrophage populations with significant numbers of CD8+ T-cells, CD4+ T-cells and NK cells throughout tumor development. For this reason, in comparison to 4T1 tumors, MC38 tumors are more responsive to immune checkpoint blockage and the addition of ΔECHy mediated HA reduction and stromal remodeling played a key role in the improved response even at a subtherapeutic dose.

### Example 10. Biocompatibility and Systemic Elimination of ΔECHy

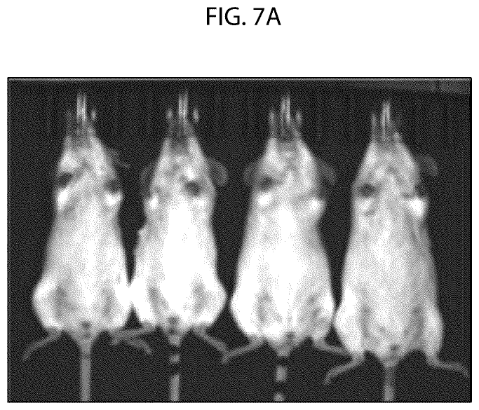

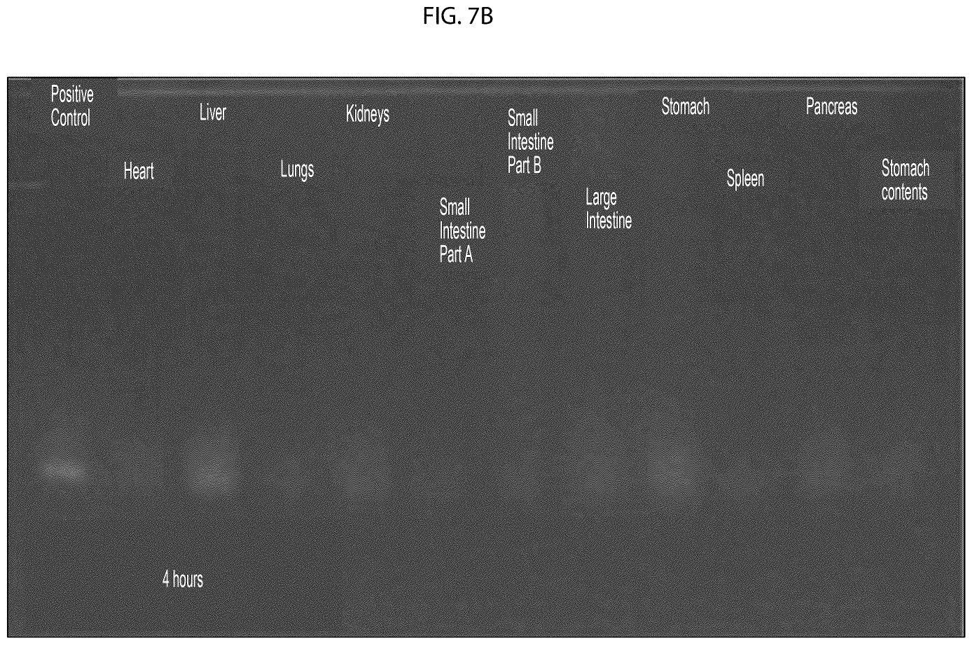

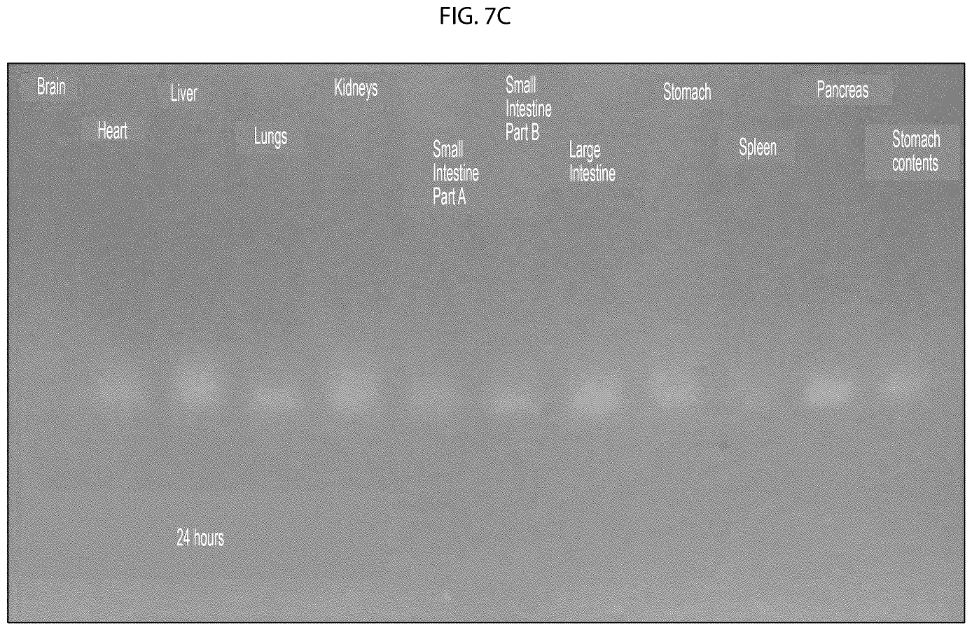

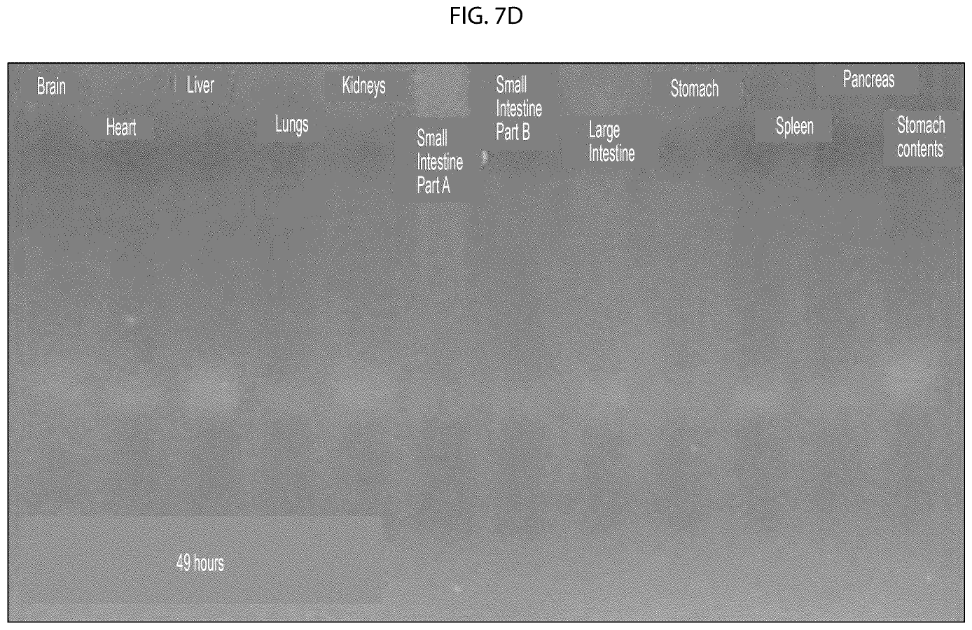

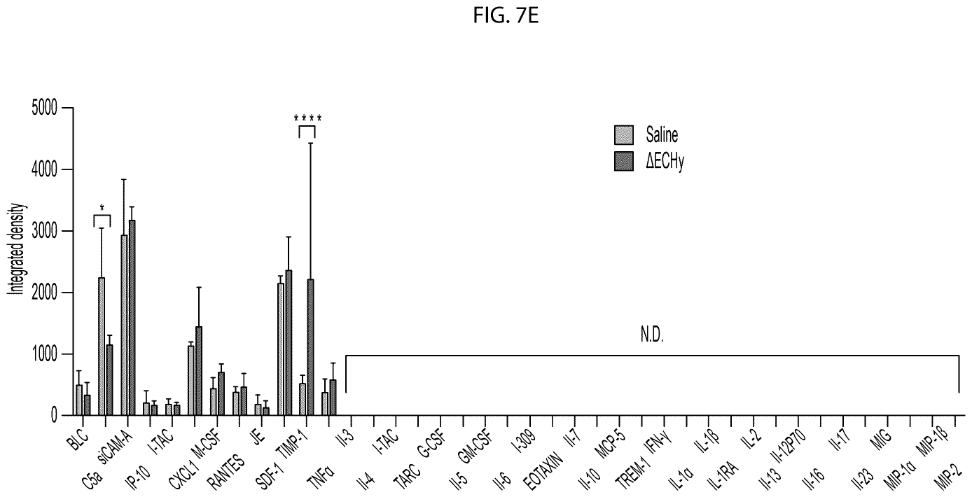

An important aspect of using a live biotherapeutic for therapy is the selection of an appropriate biocompatible and non-toxic cellular carrier. Ideally, when used for therapeutic applications, the engineered bacteria should exclusively populate tumors and be cleared from the whole system as the therapy progresses towards completion. Most importantly, neither the bacteria nor the degradation products should elicit a detrimental immune response towards the heathy tissues. For fulfilling this aim, a trial was conducted for analyzing ΔECHy for its distribution and immune response in-vivo. To better understand the in vivo biodistribution and fate of ΔE naive BALB/cJ mice were administered ΔElux intravenously and imaged after a period of 24 hours for bioluminescent signals. No BLI signals were observed from the mice which would indicate any discernable presence of ΔElux. Thus, it is likely that majority of the bacteria are eliminated within 24 hours (FIG. 7A). BALB/cJ mice are immunocompetent and their immune system is capable of clearing the ΔE strains from the circulation quite easily. Any persistent ΔE in circulation or in the organs could be in very low numbers for repopulation. Nevertheless, at the end of 24 hours, ΔE population is certainly below the detection limits of the bioluminescent imaging system.

A more sensitive method for detecting the presence of ΔE or the presence of ΔE genomic DNA is to analyze different tissue samples for the presence of genomic components via PCR based amplification. The study was repeated in naive BALB/cJ mice and collected different organs at the end of 4, 24 and 49 hours respectively. The organs were processed for the isolation of total genomic DNA. The genomic contents were analyzed at each time point via PCR based amplification with primers against the pakgfplux luciferin-luciferase plasmid (pakgfplux1). The amplified reaction products were analyzed by nucleic acid gel electrophoresis. At the end of 4 hours a faint band was observed in the liver and stomach (FIG. 7B). However, at the end of 24 hours faint bands were also observed in various organs of the digestive tract, heart, liver, lungs and the kidneys (FIG. 7C). This could either be due to presence of ΔElux or due to the residual plasmid/genomic contents or their fragments in circulation. 49 hours post intravenous injection the bands started to become weaker (FIG. 7D) and disappear eventually. Thus, the in vivo study in healthy mice shows initial rapid elimination which then gradually declines over time. The remaining small number of bacteria are cleared slowly, and as shown through PCR analysis there was no trace of bacteria in the body except in the tumor.

Analyzing the different markers of immune response and inflammatory mediators is expected to provide a better picture of ΔE biocompatibility. ΔECHy was administered to naive BALB/cJ mice systemically via intravenous injection and measured 40 major cytokines and chemokines in plasma after a period of 24 hours with a membrane-based antibody sandwich immunoassay. The 24-hour period is a satisfactory time point to measure any acute immune reaction since the biodistribution study showed that the majority of the administered dose is cleared within that time frame. An array plot data from the immunoassay was quantitated to generate a protein profile histogram for the detected proteins (FIG. 7E, 16A, 16B). Among all cytokines and chemokines, only TIMP-1 (p<0.0001) was found to be significantly upregulated and C5a (p=0.0160) downregulated. Complement 5a or C5a is a part of the complement/clotting cascade. Tissue inhibitor of metalloproteinases or TIMP-1 is a metalloproteinase inhibitor and also a signaling cytokine known to attenuate chronic pain. The increased presence of TIMP-1 might be related to the method of asphyxiation induced euthanasia or because of the stress induced during submandibular vein blood collection. Therefore, the cytokine profiling study does not indicate any meaningful changes in comparison to the controls which would suggest there is no acute inflammatory and immune responses against ΔECHy. These results indicate that ΔECHy is cleared rapidly, does not induce any off-target toxicity nor has any detrimental effect towards animal health in mouse models. Results further indicate that ΔECHy appears to be safe for in vivo administration and therapy.

Aspects of the present disclosure can be described with reference to the following numbered clauses, with preferred features laid out in dependent clauses.
1. The first aspect relates to a hypervesiculating Escherichia coli bacterium engineered to produce outer membrane vesicles (OMVs), said OMVs packaging a fusion peptide comprising cytolysin A (ClyA) and hyaluronidase (Hy).
2. The second aspect, alone or in combination with the first aspect, relates to a hypervesiculating Escherichia coli bacterium, wherein the Escherichia coli is Escherichia coli Nissle.
3. The third aspect, alone or in combination with any of the previous aspects, relates to a hypervesiculating Escherichia coli bacterium, wherein the bacterium is engineered to produce OMVs by replacing an n1pI gene sequence with a chloramphenicol cassette.
4. The fourth aspect, alone or in combination with any of the previous aspects, relates to a hypervesiculating Escherichia coli bacterium of claim 1, wherein the fusion peptide has an amino acid sequence with at least 75%, at least 80%, at least 85%, at least 90%, at least 95%, at least 98%, or at least 99% sequence identity to SEQ ID NO:17.
5. The fifth aspect, alone or in combination with any of the previous aspects, relates to an Escherichia coli Nissle (ΔECHy) bacterium engineered to produce outer membrane vesicles (OMVs), said OMVs packaging a fusion peptide, said fusion peptide having at least 95% sequence identity to SEQ ID NO:17.
6. The sixth aspect, alone or in combination with any of the previous aspects, relates to an outer membrane vesicle (OMV) comprising a fusion peptide with an amino acid sequence with at least 95% sequence identity to SEQ ID NO:17, wherein said outer membrane vesicle is isolated from a hypervesiculating Escherichia coli bacterium.
7. The seventh aspect, alone or in combination with any of the previous aspects, relates to a method of remodeling a tumor, the method comprising contacting the tumor with a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).
8. The eighth aspect, alone or in combination with any of the previous aspects, relates to a method wherein the tumor is a hypoxic tumor.
9. The ninth aspect, alone or in combination with any of the previous aspects, relates to a method, wherein the ΔECHy bacterium localizes to the tumor.
10. The tenth aspect, alone or in combination with any of the previous aspects, relates to a method wherein the OMVs produced by the ΔECHy bacterium deliver the fusion peptide to a microenvironment of the tumor.
11. The eleventh aspect, alone or in combination with any of the previous aspects, relates to a method wherein the ΔECHy bacterium degrades extracellular hyaluronic acid in the tumor microenvironment.
12. The twelfth aspect, alone or in combination with any of the previous aspects, relates to a method wherein the method induces stromal changes to the tumor that permit penetration of a second therapeutic agent to the tumor microenvironment.
13. The thirteenth aspect, alone or in combination with any of the previous aspects, relates to a method, wherein the method promotes tumor cell death.
14. The fourteen aspect, alone or in combination with any of the previous aspects, relates to a method, wherein the method is in vivo or in vitro.
15. The fifteenth aspect, alone or in combination with any of the previous aspects, relates to a method of treating a solid tumor in a subject in need thereof, the method comprising administering to the subject an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).
16. The sixteenth aspect, alone or in combination with any of the previous aspects, relates to a method wherein the subject is a mammal.
17. The seventeenth aspect, alone or in combination with any of the previous aspects, relates to a method wherein the subject is a human.
18. The eighteenth aspect, alone or in combination with any of the previous aspects, relates to a method wherein the tumor is a hypoxic tumor.
19. The nineteenth aspect, alone or in combination with any of the previous aspects, relates to a method wherein the ΔECHy bacterium is administered orally, parenterally, or intratumorally.
20. The twentieth aspect, alone or in combination with any of the previous aspects, relates to a method wherein the ΔECHy bacterium localizes to the tumor.
21. The twenty-first aspect, alone or in combination with any of the previous aspects, relates to a method wherein the OMVs produced by the ΔECHy bacterium deliver the fusion peptide to a microenvironment of the tumor.
22. The twenty-second aspect, alone or in combination with any of the previous aspects, relates to a method wherein the ΔECHy bacterium degrades extracellular hyaluronic acid in the tumor microenvironment.
23. The twenty-third aspect, alone or in combination with any of the previous aspects, relates to a method wherein the method induces stromal changes to the tumor that permit penetration of a second therapeutic agent to the tumor microenvironment.
24. The twenty-fourth aspect, alone or in combination with any of the previous aspects, relates to a method wherein the method promotes tumor cell death.
25. The twenty-fifth aspect, alone or in combination with any of the previous aspects, relates to a method further comprising administering a second therapeutic agent.
26. The twenty-sixth aspect, alone or in combination with any of the previous aspects, relates to a method wherein the second therapeutic agent is selected from the group consisting of tyrosine kinase inhibitors, immune checkpoint antibodies, chemotherapy drugs, PARP inhibitors, cancer growth blockers, anti-angiogenics, immune checkpoint antibodies, other monoclonal antibodies, cell based therapies such as CAR-T, NK-T, stem cells, and oncolytic viruses, and combinations thereof.
27. The twenty-seventh aspect, alone or in combination with any of the previous aspects, relates to a method wherein the ΔECHy bacterium and the second therapeutic agent are administered concurrently or sequentially.
28. The twenty-eighth aspect, alone or in combination with any of the previous aspects, relates to a pharmaceutical composition comprising an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy); and at least one pharmaceutically-acceptable carrier.
29. The twenty-ninth aspect, alone or in combination with any of the previous aspects, relates to a pharmaceutical composition further comprising an effective amount of a second therapeutic agent.
30. The thirtieth aspect, alone or in combination with any of the previous aspects, relates to a pharmaceutical composition pharmaceutical composition wherein the second therapeutic agent is selected from the group consisting of tyrosine kinase inhibitors, immune checkpoint antibodies, chemotherapy drugs, PARP inhibitors, cancer growth blockers, anti-angiogenics, immune checkpoint antibodies, other monoclonal antibodies, cell based therapies such as CAR-T, NK-T, stem cells, and oncolytic viruses, and combinations thereof.
31. The thirty-first aspect, alone or in combination with any of the previous aspects, relates to a pharmaceutical composition wherein the fusion peptide is encapsulated within the OMVs produced by the ΔECHy bacterium.
32. The thirty-second aspect, alone or in combination with any of the previous aspects, relates to a fusion peptide having an amino acid sequence with 95% sequence identity with SEQ ID NO: 17.
33. The thirty-third aspect, alone or in combination with any of the previous aspects, related to a fusion peptide having an amino acid sequence represented by SEQ ID NO:17.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A hypervesiculating Escherichia coli bacterium engineered to produce outer membrane vesicles (OMVs), said OMVs packaging a fusion peptide comprising cytolysin A (ClyA) and hyaluronidase (Hy).

2. The hypervesiculating Escherichia coli bacterium of claim 1, wherein the Escherichia coli is Escherichia coli Nissle.

3. The hypervesiculating Escherichia coli bacterium of claim 1, wherein the bacterium is engineered to produce OMVs by replacing an n1pI gene sequence with a chloramphenicol cassette.

4. The hypervesiculating Escherichia coli bacterium of claim 1, wherein the fusion peptide has an amino acid sequence with at least 75% sequence identity to SEQ ID NO:17.

5. The hypervesiculating Escherichia coli bacterium of claim 1, wherein the fusion peptide has an amino acid sequence with at least 95% sequence identity to SEQ ID NO:17.

6-14. (canceled)

15. A method of treating a solid tumor in a subject in need thereof, the method comprising administering to the subject an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy).

16. The method according to claim 15, wherein the subject is a mammal.

17. The method according to claim 16, wherein the subject is a human.

18. The method according to claim 17, wherein the tumor is a hypoxic tumor.

19. The method according to claim 18, wherein the ΔECHy bacterium is administered orally, parenterally, or intratumorally.

20. The method according to claim 19, wherein the ΔECHy bacterium localizes to the tumor.

21. The method according to claim 15, wherein the OMVs produced by the ΔECHy bacterium deliver the fusion peptide to a microenvironment of the tumor.

22. The method according to claim 21, wherein the ΔECHy bacterium degrades extracellular hyaluronic acid in the tumor microenvironment.

23. The method according to claim 21, wherein the method induces stromal changes to the tumor that permit penetration of a second therapeutic agent to the tumor microenvironment.

24. The method according to claim 15, wherein the method promotes tumor cell death.

25. The method according to claim 15, further comprising administering a second therapeutic agent.

26. The method according to claim 25, wherein the second therapeutic agent is selected from the group consisting of tyrosine kinase inhibitors, immune checkpoint antibodies, chemotherapy drugs, PARP inhibitors, cancer growth blockers, anti-angiogenics, immune checkpoint antibodies, other monoclonal antibodies, cell based therapies such as CAR-T, NK-T, stem cells, and oncolytic viruses, and combinations thereof.

27. The method according to claim 26, wherein the ΔECHy bacterium and the second therapeutic agent are administered concurrently or sequentially.

28. A pharmaceutical composition comprising:
an effective amount of a hypervesiculating Escherichia coli Nissle (ΔECHy) bacterium engineered to produce outer membrane vesicles (OMVs), said OMVs packaging a fusion peptide comprising cytolysin A (ClyA) and hyaluronidase (Hy); and
at least one pharmaceutically-acceptable carrier.

29. The pharmaceutical composition according to claim 28, further comprising an effective amount of a second therapeutic agent.

30. The pharmaceutical composition according to claim 29, wherein the second therapeutic agent is selected from the group consisting of tyrosine kinase inhibitors, immune checkpoint antibodies, chemotherapy drugs, PARP inhibitors, cancer growth blockers, anti-angiogenics, immune checkpoint antibodies, other monoclonal antibodies, cell based therapies such as CAR-T, NK-T, stem cells, and oncolytic viruses, and combinations thereof.

31. The pharmaceutical composition according to claim 28, wherein the fusion peptide is encapsulated within the OMVs produced by the ΔECHy bacterium.


SEQUENCE LISTING (17 sequences)

SEQ ID NO: 1 - DNA, 51 bp, synthetic construct
  source 1..51: mol_type = other DNA
tagcagctgcaaagaggagaaacaccatatgacagagattgttgcggacaa

SEQ ID NO: 2 - DNA, 60 bp, synthetic construct
  source 1..60: mol_type = other DNA
cggtacccacgtggggcccgtcgactctagaggatcctcacgcaggtgcaattgtcgtta

SEQ ID NO: 3 - DNA, 60 bp, synthetic construct
  source 1..60: mol_type = other DNA
agagtcgacgggccccacgtgggtaccgtttaaaccccgggtgtacaagtactaagcttc

SEQ ID NO: 4 - DNA, 23 bp, synthetic construct
  source 1..23: mol_type = other DNA
atggtgtttctcctctttgcagc

SEQ ID NO: 5 - DNA, 60 bp, synthetic construct
  source 1..60: mol_type = other DNA
ctattgctggtccgattctgccaggtcatcttggtcctggcccagttacgccccgccctg

SEQ ID NO: 6 - DNA, 60 bp, synthetic construct
  source 1..60: mol_type = other DNA
atgaagccttttttgcgctggtgtttcgttgcgacagcatgatcggcacgtaagaggttc

SEQ ID NO: 7 - DNA, 21 bp, synthetic construct
  source 1..21: mol_type = other DNA
catgtagtacgtgtgcctcaa

SEQ ID NO: 8 - DNA, 22 bp, synthetic construct
  source 1..22: mol_type = other DNA
aacctggcctatttccctaaag

SEQ ID NO: 9 - DNA, 20 bp, synthetic construct
  source 1..20: mol_type = other DNA
agcgaaagagatttgccgga

SEQ ID NO: 10 - DNA, 20 bp, synthetic construct
  source 1..20: mol_type = other DNA
acgccactgccctttattga

SEQ ID NO: 11 - DNA, 20 bp, synthetic construct
  source 1..20: mol_type = other DNA
cttggtgggcgacattaaga

SEQ ID NO: 12 - DNA, 22 bp, synthetic construct
  source 1..22: mol_type = other DNA
cgacctctgggacttcaaataa

SEQ ID NO: 13 - DNA, 20 bp, synthetic construct
  source 1..20: mol_type = other DNA
ataataccgcgccacatagc

SEQ ID NO: 14 - DNA, 19 bp, synthetic construct
  source 1..19: mol_type = other DNA
ttgacagctagctcagtcc

SEQ ID NO: 15 - DNA, 20 bp, synthetic construct
  source 1..20: mol_type = other DNA
ataccaaaagcgccacggta

SEQ ID NO: 16 - DNA, 29 bp, synthetic construct
  source 1..29: mol_type = other DNA
ccatctaattcaacaagaattgggacaac

SEQ ID NO: 17 - protein, 537 aa, synthetic construct
MTEIVADKTVEVVKNAIETADGALDLYNKYLDQVIPWQTFDETIKELSRFKQEYSQAASVLVGDIKTLLMDSQDKYFEATQTVYEWCGVATQLLAAYILLFDEYNEKKASAQKDILIKVLDDGITKLNEAQKSLLVSSQSFNNASGKLLALDSQLTNDFSEKSSYFQSQVDKIRKEAYAGAAAGVVAGPFGLIISYSIAAGVVEGKLIPELKNKLKSVQNFFTTLSNTVKQANKDIDAAKLKLTTEIAAIGEIKTETETTRFYVDYDDLMLSLLKEAAKKMINTCNEYQKRHGKKTLFEVPEVGGGGGENLYFQGGGGGGAGENGATTTFDGPVAAERFSADTTLEAAFLKTTSETNHAATIYQAGTSGDGAALNVISDNPGTSAMYLSGTETARGTLKITHRGYADGSDKDAAALSLDLRVAGTAAQGIYVTATNGPTKGNLIALRNNTGLDDFVVKGTGRIGVGIDRAATPRAQVHIVQRGDALAALLVEGSVRIGNAATVPTSVDSSGGGALYASGGALLWRGSNGTVTTIAPA